(19) United States
(12) Patent Application Publication
(10) Pub. No.: US 2022/0383991 A1
(43) Pub. Date: Dec. 1, 2022

(54) SYSTEMS AND METHODS FOR MONOCLONAL ANTIBODY NOMOGRAMS

(71) Applicants: Diane R. Mould, Fort Myers, FL (US); Steve Molnar, Fort Myers, FL (US)

(72) Inventors: Diane R. Mould, Fort Myers, FL (US); Steve Molnar, Fort Myers, FL (US)

(21) Appl. No.: 17/829,075

(22) Filed: May 31, 2022

Related U.S. Application Data
(60) Provisional application No. 63/194,987, filed on May 29, 2021.

(51) Int. Cl. G16C 20/30; A61K 39/395

(52) U.S. Cl. CPC G16C 20/30; A61K 39/3955; A61K 2039/545

(57) ABSTRACT

Provided herein are systems and methods for constructing and using nomograms for adjustment of dosing regimens. The nomograms use measured drug concentration data to determine a specific patient's effective half-life for a drug or set of drugs. The patient-specific effective half-life is used to determine the time at which the drug concentration in the patient's body will reach a target concentration after administration of a dose. Label dosages and dosing intervals are based on an average patient, so adjustment of a dosing regimen for a specific patient better accounts for the patient's unique pharmacokinetic interaction with the drug.

### CROSS-REFERENCE TO RELATED APPLICATIONS

This application claims the benefit of priority under 35 U.S.C. § 119(e) from U.S. Provisional Application Ser. No. 63/194,987 filed May 29, 2021, the contents of which are hereby incorporated by reference in their entirety.

### TECHNICAL FIELD

This disclosures generally relates to the use of nomograms for adjustment of dosing regimens, including, without limitation, adjustment of monoclonal antibody dosing regimens. Computerized systems and methods that use pharmacokinetic models may be used to estimate pharmacokinetic parameters and to predict and propose dosing regimen adjustments for a specific patient.

### BACKGROUND

A physician's decision to start a patient on a medication-based treatment regimen involves determination of a dosing regimen for the medication to be prescribed. Different dosing regimens will be appropriate for different patients having differing patient factors, such as age, weight, health risk factors, and others. Dosing quantities, dosing intervals, treatment duration and other variables may be varied. Although a proper dosing regimen may be highly beneficial and therapeutic, an improper dosing regimen may be ineffective or deleterious to the patient's health. Further, both underdosing and overdosing can result in a loss of time, money and/or other resources, and increases the risk of undesirable outcomes.

In current clinical practice, the physician typically prescribes a dosing regimen based on dosing information contained in the package insert (PI) of the prescribed medication. In the United States, the contents of the PI are regulated by the Food and Drug Administration (FDA), and in Europe by the European Medicines Agency (EMA). As will be appreciated by those skilled in the art, the PI is typically a printed informational leaflet including a textual description of basic information that describes the drug's appearance and the approved indications and uses of the drug. Further, the PI typically describes how the drug works in the body and how it is metabolized. The PI also typically includes statistical details based on trials regarding the percentage of people who have side effects of various types, interactions with other drugs, contraindications, special warnings, how to handle an overdose, and extra precautions. PIs also include dosing information. Such dosing information typically includes information about dosages for different conditions or for different populations, like pediatric and adult populations. Typical PIs provide dosing information as a function of certain limited patient factor information. Such dosing information is often used as a reference point for physicians in prescribing a dosage for a particular patient.

Dosing information is often developed by the medication's manufacturer, after conducting clinical trials involving administration of the drug to a population of test subjects, carefully monitoring the patients, and recording of clinical data associated with the clinical trial. The clinical trial data is subsequently compiled and analyzed to develop the dosing information for inclusion in the PI. The typical dosing information is a generic reduction or composite from data gathered in clinical trials of a population, including individuals having various patient factors, that is deemed to be suitable for an “average” patient having “average” factors and a “moderate” level of disease, without regard to specific patient's factors, including some patient factors that may have been collected and tracked during the clinical trial. By way of example, based on clinical trial data gathered for Abatacept, an associated PI provides indicated dosing regimens with a very coarse level of detail-such as 3 weight ranges (<60 kg, 60-100 kg, and >100 kg) and associated indicated dosing regimens (500 mg, 750 mg, and 1000 mg, respectively). Such a coarse gradation linked to limited patient factors (e.g., weight) ignores many patient-specific factors that could impact the optimal or near optimal dosing regimen. Accordingly, it is well-understood that a dosing regimen recommended by a PI is not likely to be optimal or near-optimal for any particular patient, but rather provides a suggested starting point for treatment, and it is left to the physician to refine the dosing regimen for a particular patient, largely through a trial and error process.

As part of that process, the physician may determine a dosing regimen for the patient as a function of the PI information. For example, for a patient having a weight falling into the 60-100 kg weight range, the indicated dosing regimen may be determined to be 750 mg, every 4 weeks. The physician then administers the indicated dosing regimen by prescribing the medication, causing the medication to be administered and/or administering a dose to the patient consistent with the dosing regimen.

As referenced above, the indicated dosing regimen may be a proper starting point for treating a hypothetical “average” patient, but the indicated dosing regimen is very likely not the optimal or near-optimal dosing regimen for the specific patient being treated, particularly after the initial dosing is completed (e.g., after completion of an induction phase of dosing in which the patient's drug concentration is quickly brought up to a therapeutic level) and the patient reaches the maintenance stage (e.g., when less frequent doses or lower doses are administered to maintain the therapeutic level of drug concentration). This may be due, for example, to the individual factors of the specific patient being treated (e.g., age, concomitant medications, other diseases, renal function, etc.) that are not captured by the factors accounted for by the PI (e.g., weight). Further, this may be due to the coarse stratification of the recommended dosing regimens (e.g., in 40 kg increments), although the proper dosing is more likely a continuously variable function of one or more patient factors.

Current clinical practice acknowledges this discrepancy. Accordingly, it is common clinical practice to follow-up with a patient after a period of time on an initial dosing regimen to reevaluate the patient and dosing regimen. Accordingly, the physician may later evaluate the patient's response to the indicated dosing regimen. However, any adjustment to the initial dosing regimen is made largely on an ad hoc basis, as part of a trial and error process, and based largely on data gathered after observing the effect on the patient of the last-administered dosing regimen.

After administering the adjusted dosing regimen, the patient's response to the adjusted dosing regimen is evaluated. The physician then again determines whether to further adjust the dosing regimen, and the process repeats. Such a trial-and-error based approach relying on generic indicated dosing regimens and patient-specific observed responses works reasonably well for medications with a fast onset of response. However, this approach is not optimal, and often not satisfactory, for drugs that take longer to manifest a desirable clinical response. Further, a protracted time to optimize dosing regimen puts the patient at risk for undesirable outcomes. In some instances, the administered dosing regimen involves too long of a dosing interval, so the patient is being administered more drug than needed based on their individual pharmacokinetic clearance. In some situations, a patient with faster pharmacokinetic clearance may need a shorter dosing interval to ensure that the concentration of drug in their body stays at a therapeutic level until the next dose is administered.

Doctors prescribing a drug often do not know two key pharmacokinetic parameters of the drug; the effective drug half-life for the patient or the maximum drug concentration in the patient's blood immediately after administration of the drug. Without knowing these two key parameters, the doctor cannot easily determine the amount of time it would take for the patient's drug concentration to reach the desired target drug concentration (e.g., 5 μg of infliximab per mL of serum). This amount of time determines the time when the patient needs the next dose of the drug, because the drug concentration should not decrease to lower than the target in order for proper maintenance of a therapeutic response.

### SUMMARY

The systems and methods disclosed herein provide a caregiver with individualized nomograms that determines the effective drug half-life for a patient, thereby allowing the caregiver to determine the amount of time for the drug concentration to reach the target concentration in the patient's body (hereinafter referred to as the “time to target”), which can guide the selection of a dosing interval or dosing amount. Knowing the time to target enables the doctor to more accurately administer doses so that the patient is not being given more or less drug than is required for maintenance of the target concentration. This more accurate, more personalized dosing is particularly advantageous for expensive drugs, such as monoclonal antibodies, such that the patient (or his or her insurance company) is not paying for more drug than is needed. For example, the average cost of a monoclonal antibody drug is about 40,000 USD per year, so a patient who has twice the average half-life can extend their dosing interval by double and save about 20,000 USD of drug per year.

Accordingly, systems, methods, and articles are disclosed herein for producing and using nomograms for dosing regimen adjustment. These nomograms are particularly advantageous for administration of monoclonal antibody drugs, such as infliximab, adalimumab, vedolizumab, and others discussed herein, which have high between-patient variance in pharmacokinetics and pharmacodynamics.

A dosing regimen (also referred to as a treatment plan) may include a schedule for dosing, one or more dosing amounts, and/or one or more routes of administration. Dosing regimens are not limited to just one drug, but can include multiple drugs, with the same or different routes of administration. A drug (also referred to as a pharmaceutical, medicine, medication, biologic, compound, treatment, therapy, or any other similar term) is a substance which has a physiological effect when introduced into a body. In some implementations, the systems described herein are not specific to a particular drug but instead apply to a class, or subset or grouping of drugs used in a drug-agnostic model. As used herein, the term “drug” may refer to a single drug or a class or set of drugs.

A class of drugs may be a group of drugs larger than one, which exhibit at least one similar pharmacokinetic (PK) and/or pharmacodynamic (PD) behavior, share a common mechanism of action, or a combination thereof (e.g., a range of drugs with differing pharmacokinetic properties but other similarities such as similar molecular weight and indication). As an example, a set of drugs may treat the same disease or be used for the same indication, examples of which include general inflammatory disease, inflammatory bowel disease (IBD, e.g. ulcerative colitis, Crohn's disease), rheumatoid arthritis, ankylosing spondylitis, psoriatic arthritis, psoriasis, rhinitis, asthma, or multiple sclerosis. A set of drugs may have a similar chemical structure. For example, a set of drugs could include monoclonal antibodies, recombinant monoclonal antibodies, murine monoclonal antibodies, chimeric monoclonal antibodies, human antibodies, monoclonal antibody fragments, or anti-inflammatory monoclonal antibodies. The methods described herein may be applied to types of drugs other than monoclonal antibodies, such as small molecules, biologics, or other drugs that may be appreciated by those skilled in the art. A user such as a doctor, clinician, or a user building a nomogram may define a class of drugs based on specific criteria, and members of that class may be electronically designated in a database as being part of that class. That database is accessible to systems and methods disclosed herein, for use in determining a class-based dosing regimen that could be used for any drug in the class. In some implementations, a dosing regimen output from the model is not specific to a single drug but is generic to the class of drugs, and suitable for any drug in that class. For example, a dosing regimen may include a drug-agnostic unit measurement (e.g., one unit, two units, three units, etc, where a unit corresponds to a specified amount of an active agent) and a time or times for administration.

Drugs may be administered through a variety of routes, such as subcutaneously, intravenously, or orally. Pharmacokinetic models may account for route of administration by taking the route of administration as a variable input to the system, allowing greater flexibility for the model. If a patient is treated with one drug (e.g. infliximab), then later treated with another drug (e.g., vedolizumab), the system may retain all patient-specific data (drug concentration measurements, clearance rates, weight measurements, etc.) from the patient's treatment on infliximab when determining an appropriate dosing regimen once the patient is being treated with the new drug. Retaining patient-specific data allows the model to accurately anticipate the patient's ability to process a drug and thereby provide more suitable, patient-specific dosing regimens when a patient changes drug therapy.

One aspect of the present invention relates to a method for constructing a nomogram useful for adjusting a dose and/or a dose interval of a dosing regimen of a drug comprising a monoclonal antibody or monoclonal antibody construct to be administered to a specific patient. The method may be computer implemented and may include the step of: receiving at an input module of a processor one or more of the following data sets: (1) data indicative of a target drug trough concentration for a specific patient, (2) data indicative of a prior dose amount of the drug previously administered to the patient, (3) data indicative of the weight of the specific patient, (4) data indicative of a current dose interval, (5) data indicative of a measured drug trough concentration in the specific patient; simulating an effective drug half-life range and a corresponding range of expected drug trough concentrations at the current dose interval based on the patient weight, a range of drug clearance values, the current dose interval, and the prior dose amount. The method may further include one or more of the steps of plotting the range of expected drug trough concentrations against the effective drug half-life range as a drug concentration curve on the nomogram; identifying the measured drug trough concentration in the specific patient on the drug concentration curve on the nomogram; determining an effective drug half-life of the specific patient based on the identified measured drug concentration on the drug concentration curve; and simulating a plurality of time-to-target values for the specific patient based on the determined drug effective half-life and the target drug trough concentration, each time-to-target value corresponding to an available dose in a plurality of available doses. Additional outputs that can be produced by the method include but are not limited to a recommended dosing interval and a recommended dose amount, each of which may be determined based on the time-to-target value(s) for the administered dose amount and target concentration.

Simulated time-to-target values may be plotted on a chart, outputted in a table, or transmitted to an output device (e.g., a physician's personal device, a healthcare system or network, or a patient's personal device). The results may be stored in a library, such as a memory device or cloud memory architecture. The library may store dose, weight, measured concentration, or any other parameters discussed herein, for each individual patient for whom a nomogram is generated. When another patient with one or more matching parameters is in need of a nomogram, the previously generated nomogram results can be looked up, rather than re-computing the nomogram process, thus saving time and computing efficiency.

In some implementations, the processor is configured with a pharmacokinetic model. Simulating the effective drug half-life range and corresponding range of expected drug trough concentrations comprises: inputting into the pharmacokinetic model the prior dose amount, the current dose interval, and the patient weight; incrementally stepping through a plurality of drug clearance values in the range of drug clearance values, using the pharmacokinetic model, to provide a plurality of expected drug trough concentrations; computing, using the pharmacokinetic model, a plurality of effective drug half-lives for the patient weight, each effective drug half-life corresponding to a drug clearance value of the plurality of drug clearance values; and outputting from the pharmacokinetic model the plurality of effective drug half-lives as the effective drug half-life range and the plurality of drug trough concentrations as the range of expected drug trough concentrations, wherein each drug trough concentration corresponds to an effective drug half-life of the plurality of effective drug half-lives. The pharmacokinetic model may be an open two-compartment model with a linear clearance and, optionally, a linear first order absorption.

In some implementations, the effective drug half-life range comprises effective half-lives between 2 days and 25 days, between 2 days and 30 days, between 2 days and 35 days, between 1 day and 40 days, or any other suitable range, e.g., depending on the drug. In some implementations, the specific patient is undergoing maintenance dosing. For example, maintenance dosing begins with a first maintenance dose after an induction dosing period is completed. In some implementations, the method further comprises plotting a region of effective drug half-lives of patients who participated in clinical trials for the drug to determine a label dosage for the drug.

In some implementations, the drug is infliximab. The prior dose amount may be 5 mg/kg infliximab. The target concentration may be between 1 μg/mL and 20 μg/mL. In other implementations, the drug is any one of adalimumab, vedolizumab, golimumab, ustekinumab, abatacept, rituximab, ixekizumab, certolizumab pegol, entanercept, dupilumab, tocilizumab, alemtuzumab, secukinumab, guselkumab, reslizumab, mepolizumab, omalizumab, benralizumab, sarilumab, risankizumab, tildrakizumab, ocrelizumab, olokizumab, and natalizumab.

In some implementations, the method further comprises generating a probability plot of a probabilities of a patient response over the effective drug half-life range. The probabilities may be determined using a logistical regression of a dataset for a patient population, the dataset comprising a patient response for each patient in the population. The dataset may further comprise an effective drug half-life for each patient in the population. For example, the patient response is one selected from the group of Crohn's disease activity index (CDAI), mucosal healing, fecal calprotectin (FCP) concentration, C-reactive protein (CRP) concentration, development of anti-drug antibodies (ADA), steroid usage, Mayo score, partial Mayo score, Harvey-Bradshaw index, and concentration of Factor VIII protein.

In some implementations, the method further comprises generating a plot of probabilities of anti-drug antibody presence over time, wherein a probability-time curve is generated for each of a set of effective drug half-life sub-ranges. The method may further comprise evaluating a time-to-first-anti-drug-antibody value for the specific patient based on the determined effective drug half-life.

In a second aspect, provided herein is a nomogram for determining a patient-specific dosing interval of a drug comprising a monoclonal antibody or a monoclonal antibody construct for a plurality of available doses, the nomogram comprising a computer-readable medium configured to perform the steps according to the method of the first aspect.

In a third aspect, provided herein is a graphical user interface comprising a nomogram constructed according to the steps of the method of the first aspect; a plurality of input boxes operatively coupled to the input module of the processor for receiving each of (1)-(5); a plurality of arrows on the nomogram, each arrow pointing to one of the identified measured drug concentration, the effective drug half-life of the specific patient, and the plurality of time-to-target values for the specific patient; and an output for displaying the plurality of time-to-target values for the specific patient for the plurality of available doses.

In a fourth aspect, provided herein is a method for determining a dose interval of a monoclonal antibody drug for a specific patient, the method comprising steps according to the method of the first aspect; and setting a new dose interval for each of the plurality of available doses of the drug for the specific patient to the plurality of time-to-target values for the specific patient. The method may further comprise, if the new dose interval is less than a standard-of-care dose interval, providing a recommendation to use Bayesian individualized dosing for the specific patient. An individualized dosing system may be used alongside the nomogram (i.e., in parallel) to compare results. The nomogram system may have intercompatibility with an individualized dosing system such that outputs from the dosing system are used as inputs to the nomogram, or vice versa. For example, pharmacokinetic parameters such as clearance and volume may be taken from the Bayesian individualized dosing system and used to more quickly determine the curves for the nomogram.

In a fifth aspect, provided herein is a method of treating any one of inflammatory bowel disease (IBD), rheumatoid arthritis (RA), juvenile idiopathic arthritis (JIA), ankylosing spondylitis (AS), psoriasis (PsO), psoriatic arthritis (PsA), multiple sclerosis (MS), atopic dermatitis, eczema, rhinitis, and asthma, with an intravenous or subcutaneous administration of a monoclonal antibody or a monoclonal antibody construct to a specific patient, the method comprising steps according to the method of the fourth aspect; and administering a new dose of the plurality of available doses of the drug to the specific patient at the corresponding new dose interval.

In a sixth aspect, provided herein is a method of rationing monoclonal antibody drug doses, the method comprising steps according to the method of the fourth aspect.

In some implementations of any of the above aspects, the model is a pharmacokinetic or a pharmacokinetic-pharmacodynamic model. The model may describe both pharmacokinetics and pharmacodynamics. Pharmacokinetic or pharmacodynamic components of the model may indicate concentration time profiles of the plurality of drugs. A pharmacokinetic component of the model may be based on clearance parameters representative of inflow and outflow of the drug(s) in the patient's body, for example, in a one or two compartment model. A pharmacodynamic component of the model may be based on synthesis and degradation rates of a pharmacodynamic marker indicative of an individual response of the patient to the drug. In some implementations, the model includes both a pharmacokinetic component and a pharmacodynamic component, and the components are interrelated. For example, the clearance of the pharmacokinetic component may be a function of the pharmacodynamic response, and/or vice versa. The model may employ Bayesian methods, such as Bayesian forecasting to predict concentration time profiles for one or more dosing regimens.

In some implementations of any of the above aspects, the method further includes receiving additional patient data indicative of a second measured concentration of the patient from administration of the specific drug according to a recommended dosing regimen. The additional patient data may comprise additional concentration data indicative of one or more concentration levels of the specific drug in one or more samples obtained from the patient. The nomogram is then updated based on the second measured concentration of the patient. At least one updated dosing regimen can be determined, using the updated nomogram, to reach the treatment objective for the patient. The at least one updated dosing regimen can be outputted for the patient (e.g., transmitted to a patient's or physician's personal device, printed as a written report, or displayed on a screen as a table or graph).

In some implementations of any of the above aspects, the nomogram is used in a clinical setting in conjunction with (for example, to cross-check the results or provide a second concentration suggestion of) a patient-specific dosing recommendation system, such as one of those described in U.S. Pat. No. 10,083,400, titled “SYSTEM AND METHOD FOR PROVIDING PATIENT-SPECIFIC DOSING AS A FUNCTION OF MATHEMATICAL MODELS UPDATED TO ACCOUNT FOR AN OBSERVED PATIENT RESPONSE” and filed Oct. 7, 2013; U.S. Patent Publication no. 2016/0300037, titled “SYSTEMS AND METHODS FOR PATIENT-SPECIFIC DOSING” and filed Apr. 8, 2016; U.S. Patent Publication No. 2019/0326002, titled “SYSTEMS AND METHODS FOR MODIFYING ADAPTIVE DOSING REGIMENS” and filed Apr. 23, 2019; and U.S. Patent Publication No. 2020/0321096, titled “SYSTEMS AND METHODS FOR DRUG-AGNOSTIC PATIENT-SPECIFIC DOSING REGIMENS” and filed Mar. 9, 2020. Each of the above patents and patent publications are hereby incorporated by reference in its entirety.

In another aspect, provided herein is a method for treating a patient with a personalized therapeutic dosing regimen determined using any combination of the above aspects. In yet another aspect, provided herein is a pharmaceutical formulation for administration to a patient, where the pharmaceutical formulation comprises an active ingredient in a dosing regimen determined using any combination of the above aspects.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1A is an example graph depicting a nomogram for infliximab dosing interval adjustment based on effective half-life and measured infliximab concentration, and FIG. 1B is a graph of the nomogram with arrows indicating a particular patient's results applied to the nomogram, according to illustrative implementations;

FIG. 2 is a flowchart showing a process for using a pharmacokinetic nomogram described herein, according to an illustrative implementation;

FIGS. 3A, 3B, and 3C are example graphs depicting infliximab nomograms generated for different patient weights, based on a drug-agnostic model, according to an illustrative implementation;

FIG. 4 is a block diagram of a system for performing the methods described herein, according to an illustrative implementation;

FIG. 5 is a block diagram depicting a pharmacokinetic model, according to an illustrative implementation;

FIG. 6 shows a system diagram of a computer network for adaptive dosing systems, according to an illustrative implementation;

FIGS. 7A-7G show example probability plots of various patient responses of interest against estimated effective half-life; FIG. 7A shows the probability of the Crohn's disease activity index (CDAI) at week 30 being 70 points less than baseline; FIG. 7B shows the probability of CDAI at week 30 being 150 points less than baseline; FIG. 7C shows the probability of mucosal healing evident at final colonoscopy; FIG. 7D shows the probability of C-reactive protein (CRP) concentration in normal range (less than 10 mg/L) at week 30; FIG. 7E shows the probability of CRP concentration in normal range (less than 10 mg/L) at week 54; FIG. 7F shows the probability of anti-drug antibody (ADA) development; and FIG. 7G shows the probability of steroid usage at week 54;

FIGS. 8A-8G show example Kaplan-Meier plots of time to first ADA (TTFADA) for various predictors; FIG. 8A shows a TTFADA Kaplan-Meier plot by estimated effective half-life; FIG. 8B shows a TTFADA Kaplan-Meier plot by sex; FIG. 8C shows a TTFADA Kaplan-Meier plot by baseline weight (BWT); FIG. 8D shows a TTFADA Kaplan-Meier plot by age; FIG. 8E shows a TTFADA Kaplan-Meier plot by Crohn's disease duration (CCDUR); FIG. 8F shows a TTFADA Kaplan-Meier plot by presence of immune-modulators (IMM); and FIG. 8G shows a TTFADA Kaplan-Meier plot by dose; and

FIGS. 9A-9C show example survivor plots of TTFADA for significant predictors; FIG. 9A shows a TTFADA survivor plot for estimated effective half-life; FIG. 9B shows a TTFADA survivor plot for age; and FIG. 9C shows a TTFADA survivor plot for IMM.

### DETAILED DESCRIPTION

The systems and methods described herein construct and use a nomogram for evaluating a specific patient's pharmacokinetic effective half-life for a drug based on measured drug concentration, and determining an appropriate dosing interval of the drug based on a calculated time to reach a target concentration of the drug in the specific patient's body (i.e., “time-to-target”). A nomogram generally refers to a mathematical tool such as a diagram or calculator that represents relationships between three or more variables. Nomograms may be graphical in form and use a geometric construction that allows a user to pinpoint a result knowing one or more of the variables. Nomograms are commonly used in various fields including chemical engineering, seismology, aeronautics, ballistics, and physiology.

In pharmacokinetics, the apparent or “effective half-life” (as used herein) is generally the rate of accumulation or elimination of a biochemical or pharmacological substance in an organism. Specifically, half-life relates to the time for drug concentration in the patient to drop by 50%. It reflects the loss of drug in the system and can be an important determinant of drug accumulation. The effective half-life reflects the cumulative effect (e.g., a weighted average) of the individual half-lives resulting from one or more of the kinetics of elimination, kinetics of absorption, kinetics of disappearance, a complex function of elimination and distribution, or a combination of the above for one or more physiological compartments. On repeated administration of a drug according to a regimen, drugs with longer effective half-lives will accumulate more slowly but to a greater extent.

The nomograms described herein (e.g., a plot, a table, or a calculator) are constructed based on (and reflect) two pharmacokinetic (PK) relationships: (1) the relationship between the drug effective half-lives and the amount of time that will pass before the target concentration is reached, and (2) the relationship between drug effective half-lives and the concentration of drug in patients over time after the previous administration. Different effective half-lives will result in different concentrations after the same number of days after dosing, depending on the drug. The exact concentration values will also vary for each day in the dosing interval after the previously administered dose. The target concentration is the lowest concentration of drug in patient serum (or blood, or tissue) that the physician deems to be allowable before giving the next dose. Effective half-lives vary for different drugs-for example, monoclonal antibody fragments have effective half-lives on the scale of hours, murine monoclonal antibodies have effective half-lives on the scale of days, chimeric monoclonal antibodies and human monoclonal antibodies have effective half-lives on the scale of weeks.

Both relationships (1 and 2 above) are calculated using the exact same effective half-life values, so their graphs are superimposable. The effective half-life values are plotted on the x-axis, the time to target values are plotted on the left y-axis, and the associated trough drug concentrations are plotted on the right y-axis; the x=0, y=0 coordinate (origin) is the lower-left corner for both relationships and represents a theoretical effective half-life of 0.

The first PK relationship (1) may be based on the following equation (Eqn. 1) which determines the time to target based on the patient's effective half-life, maximum concentration, and the target concentration:

$$Time to Target = [ Half Life ] \times [ \frac{ln ( Target Conc . / Max . Conc . )}{- ln ( 2 )} ] \qquad Eqn . 1$$

The time to target in Eqn. 1 will be different for any different drug target level selected by a physician. Nomograms may also be constructed for any drug targets, or configured to allow a user to select a target (e.g., by entering a target concentration value or selecting from a list of recommended targets). The time to target will be different for different maximum concentrations, which relates to the dose amount (i.e., what is provided to the patient). Body weight may be taken into account during construction of the nomogram by using a modified PK model.

The patient's effective half-life typically changes during induction dosing, so a physician may decide to use the nomogram once the patient starts maintenance dosing and the effective half-life has stabilized. According to Eqn. 1, patients having short effective half-lives will have short time-to-target dosing intervals. In the case of infliximab or other drugs, regardless of the target infliximab (or other drug) concentration, patients with time to target values less than standard-of-care dosing intervals may be considered for more individualized dosing, for example, using the dosing regimen recommendation systems described herein.

The second PK relationship (2) may be based on the following equation (Eqn. 2) which determines a patient's infliximab concentration at the “#” (number) of days based on the patient's drug effective half-life and maximum concentration:

$$Conc . at \# of days = [ Max . Conc . ] \times [ \frac{1}{2} ∧ ( \frac{\# of days}{Half Life} ) ] \qquad Eqn . 2$$

The drug concentration can be calculated for any day in the dosing interval. The amount of drug affects the maximum concentration, so the number of days will be different for different dose amounts for the same given target. Similar to Eqn. 1, the effective half-life often changes significantly during induction, over about the first six weeks of dosing, so the nomogram may be best constructed so as to be used during maintenance rather than during induction.

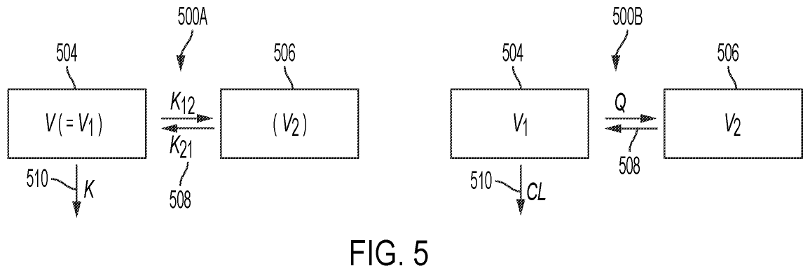

However, the doctor likely will not know the specific patient's effective half-life or maximum concentration for infliximab and thus cannot calculate Eqns. 1 and 2 exactly for the specific patient. So, according to the methods described herein Eqns. 1 and 2 are calculated over the entire range of drug effective half-life values for the clinical patient population. The range of drug effective half-lives may be taken from literature or collected from physicians having observed a variety of effective half-lives in patients treated with the drug. The resulting values are plotted in a Cartesian plane or tabulated to create the nomogram, and the nomogram used to determine a dose interval by identifying the appropriate effective half-life, and then identifying based on the appropriate effective half-life, the effective time-to-target. The calculation of Eqns. 1 and 2 can be performed using a pharmacokinetic model, such as the model described in relation to FIG. 5.

The methods for constructing nomograms described herein can be applied to dosing regimen adjustments of any pharmaceutical drug, including but not limited to monoclonal antibodies. The nomograms may apply to individual drugs or classes of drugs. For example, a nomogram may be used for a group of drugs having similar pharmacokinetic properties.

Examples of such drugs are included in Table 1. The following definitions are used in Table 1: “iv” is intravenous; “sc” is subcutaneous; “RA” is rheumatoid arthritis; “AS” is ankylosing spondylitis; “UC” is ulcerative colitis; “CD” is Crohn's disease; “IBD” is inflammatory bowel disease, which may include ulcerative colitis and/or Crohn's disease; “PsO” is psoriasis; “PsA” is psoriatic arthritis; and “MS” is multiple sclerosis. In some implementations, the systems described herein may not be specific to a particular drug but instead apply to a class, or other subset or grouping of drugs (e.g., drugs that are expected to have a similar pharmacokinetic or pharmacodynamic effect, drugs known to be candidates of treating a particular condition, or other point of similarity).

TABLE 1
Examples of drugs that may be used in development of a dosing nomogram described herein.

| Generic | Brand | | | | |
|---|---|---|---|---|---|
| Name | Name | Mechanism | Source | Indication | Route |
| infliximab | Remicade | anti-tnf | chimeric | RA AS IBD PsO PsA | iv |
| adalimumab | Humira | anti-tnf | human | RA AS IBD PsO PsA | sc |
| vedolizumab | Entyvio | anti α4β7 integrin | chimeric | IBD | iv |
| ustekinumab | Stelara | anti-IL-12 and IL-23 | human | IBD PsO PsA | iv sc |
| golimumab | Simponi | anti-tnf | human | RA AS PsA UC | sc |
| abatacept | Orencia | anti-CD80/CD86 | fusion | RA | iv sc |
| rituximab | Rituxan | anti-CD20 B Cell | chimeric | RA | iv |
| ixekizumab | Taltz | anti-IL-17 | chimeric | PsO PsA | sc |
| certolizumab | Cimzia | anti-tnf | fab | RA AS PsA UC | sc |
| pegol | | | fragment | | |
| etanercept | Enberel | anti-tnf | fusion | RA PsA | sc |
| dupilumab | Dupixant | anti IL4 receptor | human | PsO atopic dermatitis | sc |
| | | | | asthma | |
| tocilizumab | Actemra | anti-IL6 | chimeric | RA | iv sc |
| alemtuzumab | Lemtrada | anti-CD52 | chimeric | MS | iv |
| secukinumab | Cosentyx | anti-IL-17 | human | PsO PsA AS | sc |
| guselkumab | Tremfya | anti-IL-23 | human | PsO | sc |
| reslizumab | Cinqair | anti-IL-5 | chimeric | asthma | iv |
| mepolizumab | Nucala | anti-IL-5 | chimeric | asthma | sc |
| omalizumab | Xolair | IgE | chimeric | Asthma, rhinitis | sc |
| benralizumab | Fasenra | CD125 | chimeric | asthma | sc |
| sarilumab | Kevzara | anti IL6 receptor | human | RA | sc |
| risankizumab | Skyrizi | anti-IL-23 | chimeric | PSO | sc |
| tildrakizumab | Pumya | anti-IL-23 | chimeric | PSO | sc |
| ocrelizumab | Ocrevus | anti-CD20 | chimeric | MS | sc |
| natalizumab | Tysabri | anti a4 integrin | chimeric | MS | iv |
| canakinumab | Ilaris | anti-IL-1β | human | cryopyrin-associated | sc |
| | | | | periodic syndromes | |
| olokizumab | under FDA | Anti-IL6 | chimeric | RA | sc |
| | review | | | | |

For example, the nomograms described herein may be constructed using a pharmacokinetic drug-agnostic model capable of being used for all biologics used in the treatment of inflammatory diseases. Such a model can be used to evaluate patient-specific pharmacokinetics for fully human monoclonal antibodies (mAbs), chimeric mAbs, humanized mAbs, fusion proteins, and mAb fragments (i.e., a range of drugs with differing pharmacokinetic properties but other similarities such as similar molecular weight and indication), such as those listed in Table 1, using the same model. The model may be used in a broad patient population, including inflammatory bowel disease, rheumatoid arthritis, psoriatic arthritis, psoriasis, multiple sclerosis, and other such diseases that arise from immune dysregulation. The development and application of drug-agnostic Bayesian models for agents in other broad drug sets (e.g., the aminoglycoside antibiotics, chemotherapeutic agents that cause low white cell counts, etc.) is similarly feasible. Drugs within the class may be administered through a variety of routes, such as subcutaneous, intravenous, oral, intramuscular, intrathecal, sublingual, buccal, rectal, vaginal, ocular, nasal, inhalation, nebulization, cutaneous, or transdermal.

Pharmacokinetic models may account for route of administration by taking the route of administration as a variable input to the system, allowing greater flexibility for the model. A computational model, such as a Bayesian model may be used to determine dosing regimen recommendations in conjunction with a nomogram. Example Bayesian models are described in U.S. Pat. No. 10,083,400, titled “SYSTEM AND METHOD FOR PROVIDING PATIENT-SPECIFIC DOSING AS A FUNCTION OF MATHEMATICAL MODELS UPDATED TO ACCOUNT FOR AN OBSERVED PATIENT RESPONSE” and filed Oct. 7, 2013; U.S. Patent Publication no. 2016/0300037, titled “SYSTEMS AND METHODS FOR PATIENT-SPECIFIC DOSING” and filed Apr. 8, 2016; U.S. Patent Publication No. 2019/0326002, titled “SYSTEMS AND METHODS FOR MODIFYING ADAPTIVE DOSING REGIMENS” and filed Apr. 23, 2019; and U.S. Patent Publication No. 2020/0321096, titled “SYSTEMS AND METHODS FOR DRUG-AGNOSTIC PATIENT-SPECIFIC DOSING REGIMENS” and filed Mar. 9, 2020. Each of the above patents and patent publications are hereby incorporated by reference in its entirety. For example, each iteration of a computerized nomogram may include a calculation or a determination of a recommended dosing regimen using the Bayesian model. When additional data is made available (such as physiological parameter data or drug concentration data obtained from the patient), another iteration of the model may be performed to determine an updated recommended dosing regimen based on the additional data. This process may be repeated any number of times to reflect any new data that describes the patient. An individualized dosing system may be used in parallel with the nomogram to compare results and assess comparative dose intervals, or in series as a source of an input to the nomogram. Example individualized dosing systems include InsightRX® precision dosing, DoseMeRx® precision dosing, and iDose® precision dosing. The nomogram system may have intercompatibility with an individualized dosing system such that outputs from the dosing system are used as inputs to the nomogram, or vice versa, and thereby serve as initial values to assist in determining dose intervals. For example, pharmacokinetic parameters such as clearance and volume may be taken as outputs from a Bayesian individualized dosing system and used to more quickly determine the curves for the nomogram and thereby ultimately to determine an appropriate dosing interval based on the nomogram.

As used herein, a “dosing regimen” includes at least one dose amount of a drug or class of drugs and a recommended schedule for administering the at least one dose amount of the drug to a patient. The dose amount may be a multiple of an available dosage unit for the drug. For example, the available dosage unit could be one pill or a suitable fraction of a pill that results when it is easily split, such as half a pill. In some implementations, the dose amount may be an integer multiple of the available dosage unit for the drug. For example, the available dosage unit could be a 10 mg injection or a capsule that cannot be split. For some routes of administration (e.g., IV and subcutaneous), a portion or a multiple of the dose strength can be administered. The recommended schedule includes a recommended time for administering a next dose of the drug to the patient, such that a predicted concentration time profile of the drug in the patient in response to the first pharmaceutical dosing regimen is at or above the target drug exposure or response level (e.g., a target drug concentration trough level) at the recommended time.

As described above, nomograms can be constructed for a set of drugs. A drug-agnostic model can be used to produce the nomogram such that it applies to multiple drugs based on shared similarities between the drugs. Because the drug-agnostic model applies to a set of drugs, rather than only a single drug, the model may retain patient-specific information when a patient is treated with multiple drugs within the set of drugs. For example, the set of drugs may include infliximab, vedolizumab, adalimumab, and other anti-inflammatory biologics. If a patient is treated with one drug (e.g. infliximab), then later treated with another drug (e.g. vedolizumab), the system may retain all patient-specific data (drug concentration measurements, clearance rates, weight measurements, etc.) from the patient's treatment on infliximab when determining an appropriate dosing regimen once the patient is being treated with the new drug. Retaining patient-specific data allows the drug-agnostic model to accurately anticipate the patient's ability to process a drug and thereby provide more suitable, patient-specific dosing regimens when a patient changes drug therapy. Because the drug-agnostic model(s) can fit to a broad range of data, with multiple routes of application, and a broad range of diseases, a model should learn about the drug and the individual patient (e.g., via Bayesian learning). Such drug-agnostic pharmacokinetic models, for example, represent a novel application of traditional population pharmacokinetic modeling. The ability to develop such a drug-agnostic pharmacokinetic (PK) model can be predicated on one or more of several factors, including: 1) a common universal structural PK model for all agents in a specific class, 2) similar effects of patient factors on the PK parameters, and 3) similar indications. Thus, the development and application of drug-agnostic Bayesian models for agents in other broad drug classes (e.g. the aminoglycoside antibiotics) is similarly feasible and will allow greater utility of a single drug-agnostic model rather than implementation of multiple models for each drug in a class.

Similarly, a drug-agnostic model can be constructed for drug classes that exhibit a commonality for the pharmacodynamic effect (the measured response of a drug). For example, many chemotherapeutic agents cause neutropenia or low white cell counts. This is a delayed response, with the lowest white cell counts generally occurring 7 to 9 days after the chemotherapy is administered. The impact of each drug on the duration, and nadir of white counts may differ but the underlying relationship between drug exposure and decrease in white cell count is structurally similar, allowing a practical drug-agnostic pharmacodynamic model to be developed for the class of chemotherapeutic agents that cause white cell decreases.

In some implementations, the model describes pharmacokinetics and pharmacodynamics. The model includes a PK component and a PD component, which may be separate within the model, or they may be interrelated. For example, the PK and PD components may be interrelated such that the effects of PK on PD and PD on PK are included in the model. The PK component can include a PK clearance parameter and the PD component includes a PD response parameter. The interrelation between the PK and PD components may be reflected by PK clearance parameter being a function of the PD response, or vice versa. One or more differential equations can be used to describe the patient response and clearances of the drugs in the patient. A PD component of the model may comprise a first differential equation and a PK component of the model comprises a second differential equation. The first differential equation may represent PD response by the patient, and the second differential equation may represent PK clearance by the patient. The first or second differential equation may include PD response and/or PK clearance.

The systems and methods described herein may output a recommended dosing regimen for a class of drugs without identifying a specific drug to administer to the patient. As used herein, a “dosing regimen” may include a dose amount of a drug and a recommended schedule for administering the dose amount to a patient. The recommended schedule includes a recommended time for administering a next dose of the drug to the patient, to achieve a predicted concentration time profile of the drug in the patient in response to the first pharmaceutical dosing regimen that is at or above a target, for example, a drug concentration trough level, at the recommended time.

A class of drugs indicates a group of drugs larger than one, which exhibit at least one similar PK or PD effect, or share a common mechanism of action, a similar structural model (e.g., a one, two, or more than two compartment model for pharmacokinetics), or some other similarity. For example, a similar PK effect may be clearances within a specific range. A similar effect may be a measured concentration within a specific range, for example, bioavailability, absorption, a white cell count, blood concentration level, or any of the biomarkers/measurements discussed herein. The specific range may be within a tenfold difference, i.e. values of 0.1 to 1 may be considered similar. The specific range may be specified by a user on the system interface. The drugs may be grouped into a class by the disease they treat, such as general inflammatory disease, or more particularly inflammatory bowel disease (IBD including ulcerative colitis and Crohn's disease), rheumatoid arthritis, ankylosing spondylitis, psoriatic arthritis, psoriasis, asthma, or multiple sclerosis. Drug class may also be based on drug structure. For example, a class may include monoclonal antibodies (mAbs), chimeric mAbs, fully human mAbs, humanized mAbs, fusion proteins, and/or mAb fragments. Classes of medications may include anti-inflammatory compounds, chemotherapeutics, corticosteroids, immunomodulators, antibiotics or biologic therapies, or any other suitable group. Drug classes may be further determined by patient population, i.e., pediatrics, geriatrics. Drug classes may also be determined by a user based on other criteria, and members of that class (or other group) may be electronically designated in a data base as being part of that class (or group). That database is accessible to systems and methods disclosed herein, for use in determining a class (or other group) based dosing regimen. A drug class or group may include variations of the same drug, such as the same drug with different routes of administration or different manufacturers. This feature may be particularly useful if a physician needs to compare generic and brand-name drugs which vary in price, availability, indication, and/or route. Many of the examples described herein are in relation to the pharmaceutical infliximab. However, the implementations described herein may apply to immunosuppressive, anti-inflammatory, antibiotic, anti-microbial, chemotherapy, anti-coagulant, pro-coagulant, anti-depressant, anti-psychotics, psychostimulants, anti-diabetic, anti-convulsant, analgesic, or any other suitable treatment.

Many of the implementations described herein relate to the treatment of IBD, such as ulcerative colitis or Crohn's disease. Although there is no standard treatment regimen for IBD, the following groups of drugs can be used to treat IBD patients: anti-inflammatory compounds, corticosteroids, immunomodulators, antibiotics or biologic therapies. One recently developed treatment includes biologic therapies (e.g., monoclonal antibodies (mAbs) such as infliximab), which target and bind to an inflammatory protein called tumor necrosis factor (TNF), rendering it inactive. In some instances, a combination of anti-TNF agents, such as infliximab, can be combined with one or more immunomodulatory agents, such as thiopurines. Such combination therapies may effectively lower elimination rates (thereby increasing drug concentration levels in a patient's blood) and reduce formation of anti-drug antibodies. The biggest challenge in treating a patient with IBD is ensuring that the patient receives adequate exposure to the treatment. The body presents several routes of “clearance” for the drugs. For example, a patient's metabolism may break down mAbs by proteolysis (breaking down of proteins), by cellular uptake, and by additional atypical clearance mechanisms associated with IBD. For example, due to the nature of the disease, patients with conditions such as focal segmental glomerulosclerosis (FSGS) often suffer from excessive losses of the drug into the urinary and or gastrointestinal tracts. Moreover, in severe IBD, mAbs are sometimes lost in feces through ulcerated and denuded mucosa, creating an additional route of clearance. Overall, IBD patients are estimated to have an infliximab elimination rate that is 40% to 50% higher than other inflammatory diseases, making IBD especially difficult to treat. The systems and methods described herein may also develop dosing regimens to treat rheumatoid arthritis, psoriatic arthritis, ankylosing spondylitis, plaque psoriasis, low levels of clotting factor VIII, hemophilia, schizophrenia, bipolar disorder, depression, bipolar disorder, infectious diseases, cancer, seizures, transplants, or any other suitable affliction.

Patient data may be used to update and refine the model for a specific patient taking a specific drug. Inputs into the systems described herein may include concentration data, physiological data, and a target response. The inputs to the model generally include concentration data, physiological data, and a target response. As discussed above, the concentration data is indicative of one or more concentration levels of a drug in one or more samples obtained from the patient, such as blood, blood plasma, urine, hair, saliva, or any other suitable patient sample. The concentration data may reflect a measurement of the concentration level of the drug itself in the patient sample, or of another analyte in the patient sample that is indicative of the amount of drug in the patient's body. The drug may be part of a treatment plan to treat a patient with a particular health condition, such as a disease or disorder like inflammatory bowel disease (IBD, including ulcerative colitis and Crohn's disease), rheumatoid arthritis, psoriatic arthritis, ankylosing spondylitis, plaque psoriasis, or any other suitable affliction. Drugs used to treat such health conditions may include monoclonal antibodies (mAbs), such as infliximab or adalimumab. While many of the examples described herein are with reference to using infliximab to treat IBD, it will be understood that the systems and methods of the present disclosure are applicable to any drug or treatment that loses its effectiveness over time in a measurable way, and may be used to treat any number of diseases, including any inflammatory disease, such as IBD.

Inputs to the system may also include other drug information, such as disease to be treated, class of drugs, route of administration, dose strength available, preferred dosing amount (e.g., 100 mg vial, 50 mg tablets, etc.), and whether the specific drug is fully human or not (e.g., chimeric). The drug information may be used to determine the available treatment options for a patient, the selected model, and the model parameters. For example, patients treated for IBD often have a higher clearance rate than those without IBD, and a drug dosing regimen for a treatment with IBD must be adjusted accordingly. The preferred dosing amount may alter a dosing regimen before the regimen is recommended for a patient. For example, if a drug is only available in 100 mg vials, the recommended dose amount may be rounded to the nearest 100 mg increment. In some implementations, the drug information excludes information identifying the drug currently used to treat the patient. For example, the drug data may be generic to a drug class. The physiological data is generally indicative of one or more measurements of at least one physiological parameter of the patient. This may include at least one of: medical record information, markers of inflammation, an indicator of drug elimination such as an albumin measurement or a measure of C-reactive protein (CRP), a measure of anti-drug antibodies, a hematocrit level, a biomarker of drug activity, weight, body size, gender, race, disease stage, disease status, prior therapy, prior laboratory test result information, concomitantly administered drugs, concomitant diseases, a Mayo score, a partial Mayo score, a Harvey-Bradshaw index, a blood pressure reading, a psoriasis area, a severity index (PAST) score, a disease activity score (DAS), a Sharp/van der Heijde score, and demographic information.

The target response may be selected by a physician based on his/her assessment of the patient's tolerance and response to drug therapy. In an example, the target response includes a target drug concentration level of a drug in a sample obtained from the patient (such as a concentration maximum, minimum, or exposure window), and may be used to determine when a patient should receive a next dose and an amount of that next dose. The target drug concentration level may include a target drug concentration trough level; a target drug concentration maximum; a target drug area under the concentration time curve (AUC); both a target drug concentration maximum and trough; a target pharmacodynamic endpoint such as blood pressure or clot time; or any suitable metric of drug exposure. The target may be decided by a physician based on the drug data and/or concentration or response. In some implementations, a target may be automatically determined by the system in order to result in a therapeutic response in the patient. The system may evaluate a plurality of targets inputted in order to determine one or more targets that result in a therapeutic response in the patient. The inputs described above (e.g., the concentration data, the physiological data, drug information, and the target response) are used by the systems and methods of the present disclosure to personalize a dosing regimen recommendation for a patient.

Based on the received inputs, the systems and methods described herein may set one or more parameter values for a computational model (such as any of the model parameters described in U.S. patent application Ser. No. 15/094,379 (the '379 application), published as U.S. Patent Application Publication No. 2016/0300037, filed Apr. 8, 2016, and entitled “Systems and Methods for Patient-Specific Dosing”, which is hereby incorporated by reference in its entirety) that generates predictions of concentration time profiles of the drug in the patient. In some implementations, the computational model is a Bayesian model. For example, the computational model may take into account historical and/or present patient data to develop a patient-specific targeted dosing regimen. As discussed in the '379 application, the computational model may comprise a pharmacokinetic component indicative of a concentration time profile of the drug, and a pharmacodynamic component based on synthesis and degradation rates of a pharmacodynamic marker indicative of the patient's individual response to the drug. The computational model may be selected from a set of computational models that best fits the received physiological data. For example, if a patient is a 45 year old man, the system may select a computational model specific to men between the ages of 30 and 50 years of age. This computational model can be individualized to a specific patient by accounting for patient-specific measurements (such as the additional concentration data and additional physiological parameter data described herein). An individualized dosing system may be used alongside the nomogram (i.e., in parallel) to compare results. The nomogram system may have intercompatibility with an individualized dosing system such that outputs from the dosing system are used as inputs to the nomogram, or vice versa. For example, pharmacokinetic parameters such as clearance and volume may be taken from the Bayesian individualized dosing system and used to more quickly determine the curves for the nomogram.

The systems and methods may rely on Bayesian analysis. For example, Bayesian analysis may be used to determine an appropriate dose needed to achieve a desirable result, such as maintaining a drug's concentration in the patient's blood near a particular level. Bayesian analysis may involve Bayesian forecasting and Bayesian updating. These Bayesian techniques may be used to develop a model that is a function not only of patient-specific characteristics accounted for in the model as covariate patient factors, but also observed patient-specific responses that are not accounted for within the models themselves, and that reflect between-subject-variability (BSV) that distinguishes the specific patient from the typical patient reflected by the model. In this manner, the present disclosure accounts for variability between individual patients that is unexplained and/or unaccounted for by traditional mathematical models (e.g., patient response that would not have been predicted based solely on the dose regimen and patient factors). Further, the present disclosure allows patient factors accounted for by typical models, such as weight, age, race, laboratory test results, etc., to be treated as continuous functions rather than as categorical (cut off) values. By doing this, the model is adapted to a specific patient, such that patient-specific forecasting and analysis can be performed, to predict, propose and/or evaluate dosing regimens that are personalized for a specific patient.

Notably, the present disclosure may be used to not only retroactively assess a dosing regimen previously administered to the patient, but also to prospectively assess a proposed dosing regimen before administering the proposed dosing regimen to the patient, or to identify dosing regimens (administered dose, dose interval, and route of administration) for the patient that will achieve the desired outcome. Bayesian forecasting process may be used to test various dosing regimens for the patient as a function of the patient's specific characteristics accounted for as patient factor covariates within the models, and the mathematical model. This forecasting involves evaluating dosing regimens based on predicted responses for a typical patient with the patient-specific characteristics. Generally, Bayesian forecasting involves using mathematical model parameters to forecast the likely response that a specific patient will exhibit with various dosing regimens. Notably, forecasting allows for determination of a likely patient response to a proposed dosing regimen before actual administration of a proposed dosing regimen. Accordingly, the forecasting can be used to test multiple different proposed dosing regimens (e.g., varying dose amount, dose interval and/or route of administration) to determine how each dosing regimen would likely impact the patient, as predicted by the patient-specific factors and/or data in the model/composite model. The forecasts may be compared to create a set of satisfactory or best dosing regimens for achieving the treatment objective or target exposure or concentration level. For example, the target may involve maintenance of a trough blood concentration level above a therapeutic threshold.

In some implementations, the recommended dosing regimen is provided with a confidence interval or prediction interval that indicates a likelihood that the particular dosing regimen will be therapeutically effective for the patient. In particular, the confidence interval or prediction interval of the projected response or concentration from the individual data may be assessed based on the complexity of the model and the amount of individual data (PK and/or PD data). In particular, the confidence interval may reflect the possible error in the individual predictions from the models.

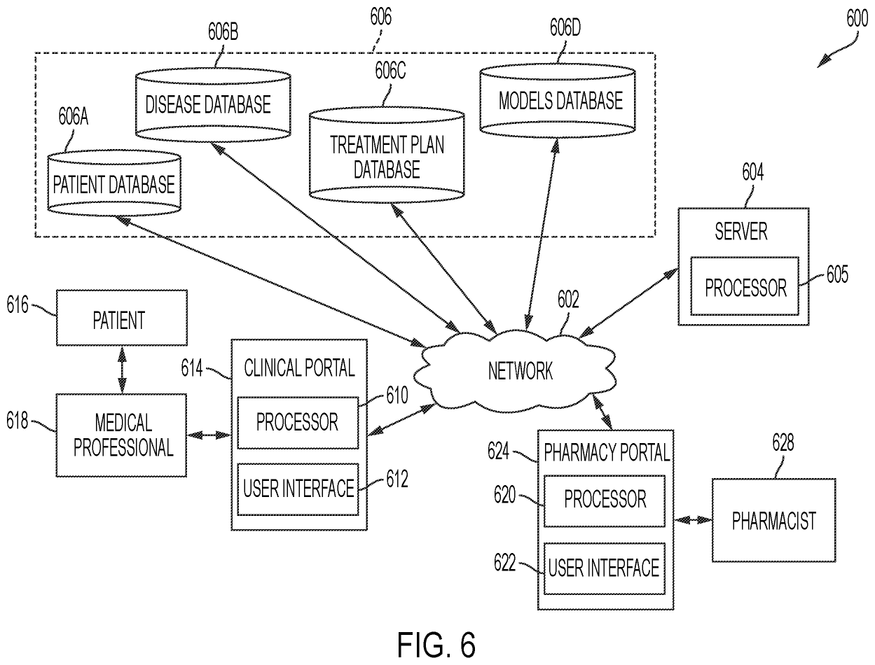

The systems and methods described herein may be used to predict patient drug clearance for a class (or other group) of drugs. Such models may be standardized to account for differences between drugs within the group of drugs. In some implementations, the model is created by collecting parameter values from a set of published models corresponding to a class of drugs. The parameter values may be collected in a lookup table. The parameter values may be converted to “standardized values” so they can be compared or pooled within the drug-agnostic model. This allows the system to simulate PK characteristics for patient populations for a published model with covariate effects as published, and for patient populations for an extended published model with all measured and presumed covariate effects. Standardized parameters may include body weight, albumin, ADA negative, presence of immune-suppressants, CRP, glucose, human or chimeric, non-IBD disease, sex, non-linear clearance, and CL. The lookup table may be used to normalize parameters to allow preliminary estimates from a drug-agnostic model. The lookup table may be manipulated by a user through a user interface, and may be stored in model database 606D of FIG. 6. The lookup table may be structured so that subsets of the table can be sent to simulation functions in a program so that each drug can be easily simulated in a variety of scenarios. Simulated concentrations from normalized parameters for each drug in the group of drugs may be compared and analyzed with respect to pooled data for that group of drugs, so as to fit the simulated concentration data to the pooled data. The drug-agnostic model for the group of drugs provides a set of parameters that applies to or is representative of all drugs in that group.

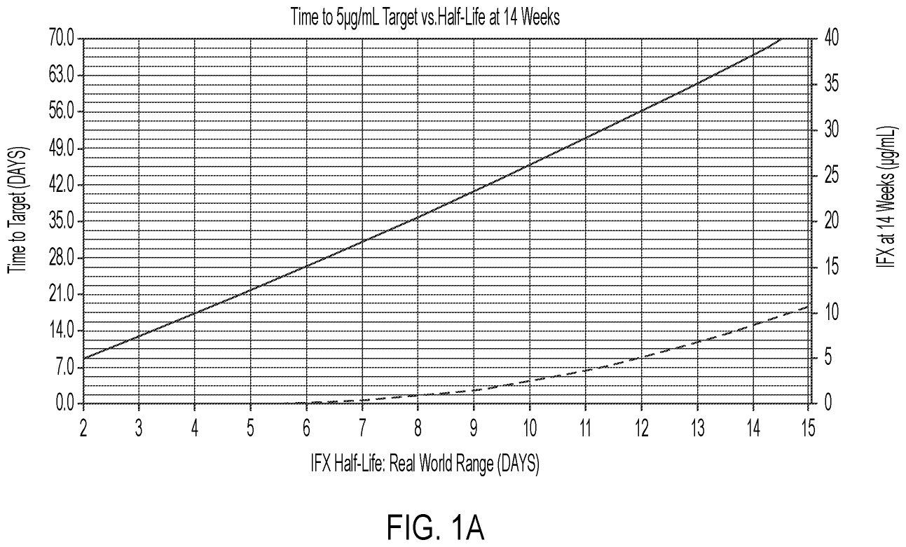

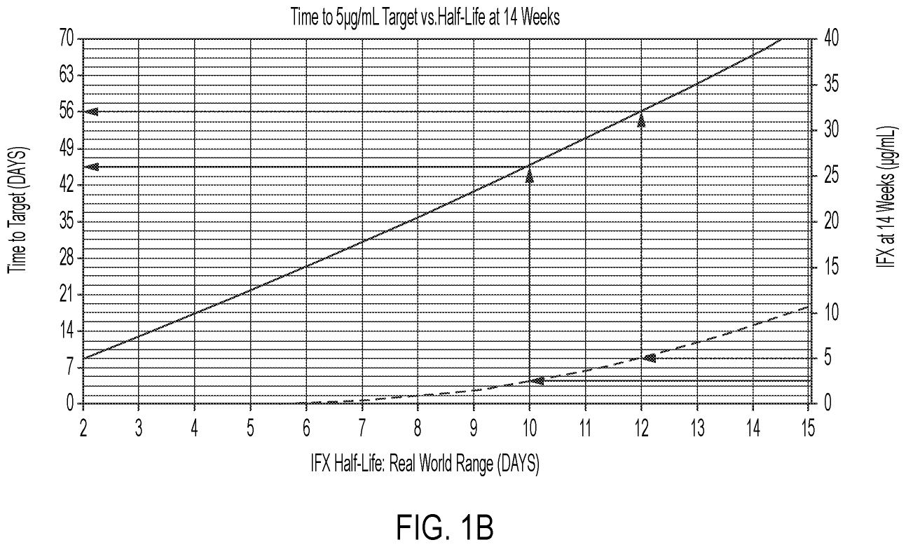

FIG. 1A shows an example nomogram for determining “time to target” of an infliximab dosing regimen based on a measured concentration of infliximab in the patient. The nomogram is constructed based on two pharmacokinetic (PK) relationships: (1) the relationship between the infliximab effective half-lives and the amount of time that will pass before the target concentration is reached, and (2) the relationship between infliximab effective half-lives and the concentration of infliximab in patients over time after the previous administration. Different effective half-lives will produce different concentrations after the same number of days after dosing. There is a different concentration curve for each day in the dosing interval. FIG. 1A shows the curve for day 56 of an 8 week dosing program. The target concentration is the lowest concentration the physician deems to be allowable before giving the next dose.

Both relationships (1 and 2 above) are calculated using the exact same effective half-life values, so their graphs are superimposable. The effective half-life values are plotted on the x-axis, the time to target values are plotted on the left y-axis, and the infliximab concentrations are plotted on the right y-axis; the x=0, y=0 coordinate (origin) is the lower-left corner for both relationships. A solid curve represents the first PK relationship (1), and a dashed curve represents the second PK relationship (2).

The first PK relationship (1) is based on Eqn. 1 (described above) which determines the time to target based on the patient's effective half-life, maximum concentration, and the target concentration. The time to target in Eqn. 1 will be different for any different target selected by a physician. In the nomogram of FIG. 1A, the target is 5 μg/mL, but nomograms may also be constructed for other targets or allow a user to select a target (e.g., 5, 7.5, or 10 μg/mL). The time to target will be different for different maximum concentrations, which relates to the dose amount (i.e., what is provided to the patient). The FDA-approved dose amount (also known as the labeled dose) of 5 mg/kg every 8 weeks is used for FIG. 1A, but the nomogram may also be constructed for other dose amounts or allow a user to select a dose amount (e.g., 5, 7.5, or 10 mg/kg). In this example, the 5 mg/kg dose amount increases the blood's infliximab concentration by 100 μg/mL regardless of patient body weight, but body weight may be taken into account during construction of the nomogram by using a modified PK model.

The patient's effective half-life changes during induction dosing, so a physician may decide to use the nomogram once the patient starts maintenance dosing and the effective half-life has stabilized. According to Eqn. 1, patient's having short effective half-lives will have short time to target dosing intervals. Regardless of the target infliximab concentration, patients with time to target values less than 28 days (4 weeks) may be considered for more individualized dosing, for example, using the dosing regimen recommendation systems described herein.

The second PK relationship (2) is based on Eqn. 2 (described above) which determines a patient's infliximab concentration at the “#” (number) of days based on the patient's drug effective half-life and maximum concentration. The infliximab concentration can be calculated for any day in the dosing interval (in this case a 56 day or 8 week dosing interval), and in the plot of FIG. 1A the concentrations are calculated for day 56. The amount of drug affects the maximum concentration, so the number of days will be different for different dose amounts for the same given target. Similar to Eqn. 1, the effective half-life changes significantly during about the first six weeks of dosing, so the nomogram may be best used during maintenance rather than induction.

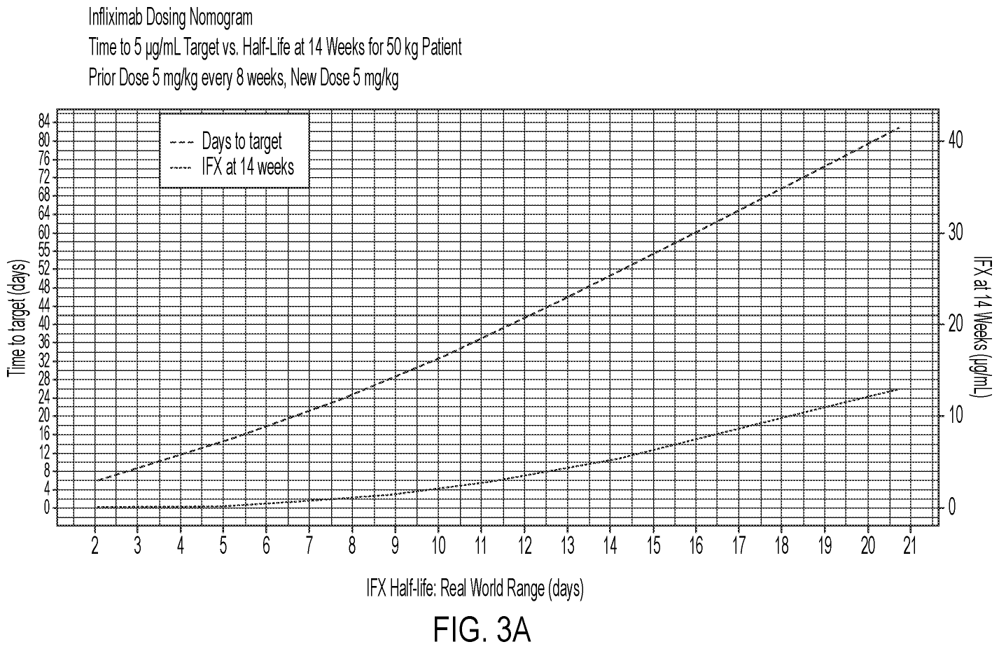

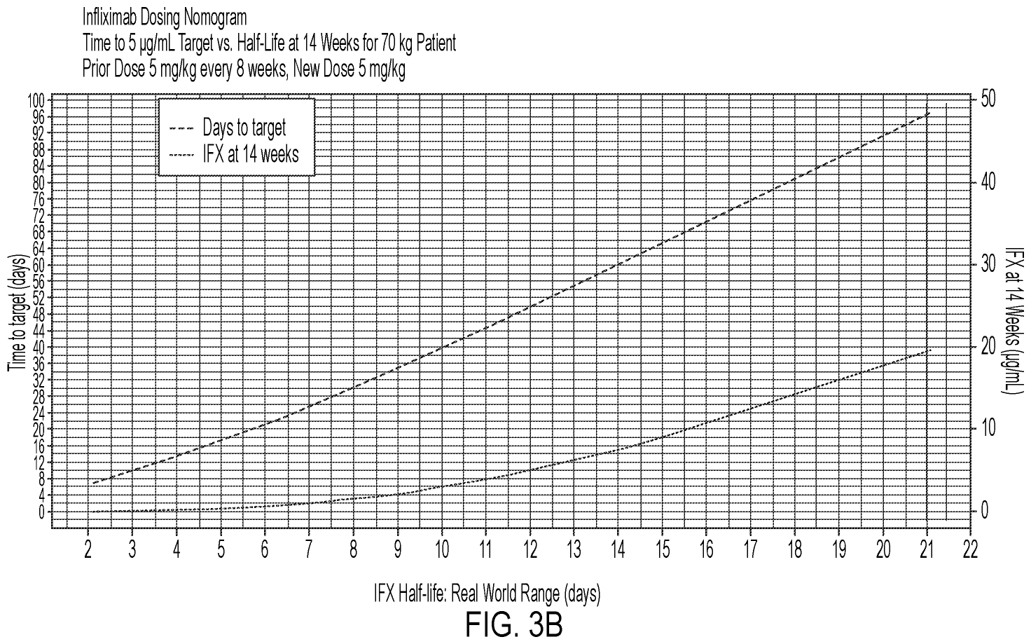

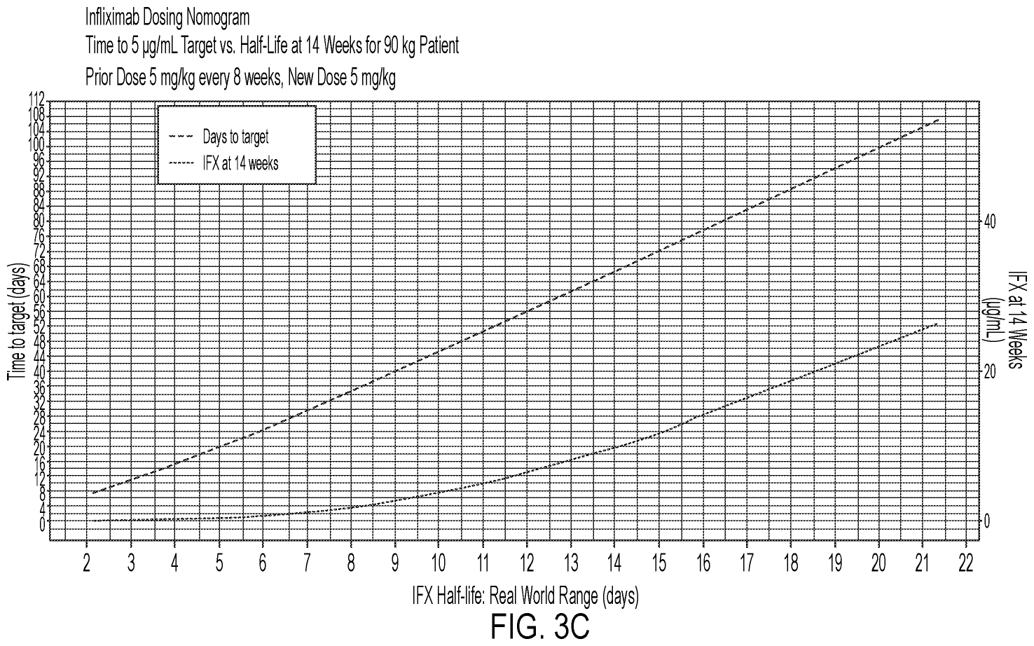

However, the doctor does not know the specific patient's effective half-life or maximum concentration for infliximab and thus cannot calculate Eqns. 1 and 2 exactly for the specific patient. To create the nomogram of FIG. 1A, Eqns. 1 and 2 are calculated over the entire range of infliximab effective half-life values for the clinical patient population. For infliximab, effective half-life values range from 2 days to 15 days. The resulting values are plotted in a Cartesian plane to create the nomogram of FIG. 1A, which represents all patients in the population 56 days after receiving a 5 mg/kg dose based on a target of 5 μg/mL (without consideration of differences in weight—the nomograms of FIGS. 3A-3C address the weight factor). The calculation of Eqns. 1 and 2 can be performed using a pharmacokinetic model, such as the model described in relation to FIG. 5.

The nomogram may be used for patients that have completed “induction” (e.g., after the first two doses on weeks 0 and 2 of treatment) and are currently undergoing “maintenance” dosing (e.g., every 8 weeks). This example nomogram is constructed for the 14^{th} week of treatment (e.g., the beginning of maintenance dosing). For infliximab, the first 3 doses (weeks 0, 2, and 6) are considered induction, because the dosing intervals are shorter than 8 weeks. At week 14, maintenance dosing begins for infliximab patients. Other monoclonal antibodies and other drugs have different induction durations. For example, adalimumab is 2 doses.

FIG. 1B depicts two examples of using the nomogram of FIG. 1A based on measured infliximab concentration data. The first example, shown with dotted arrows, represents a blood sample being taken on the 56^{th} day after receiving a 5 mg/kg dose (the labeled dose for infliximab), and laboratory testing measures an infliximab concentration of 5 μg/mL in the blood sample. Using the nomogram starting at the right y-axis, the measured infliximab concentration is plotted as a dotted arrow to the dashed curve (representing the PK relationship between concentration and effective half-life). This reveals that the specific patient has an infliximab effective half-life of 12 days. Now knowing the patient's effective half-life, the corresponding time to target is revealed by continuing the dotted arrow up to the solid curve (representing the PK relationship between effective half-life and time to target) and then to the left y-axis. The time-to-target for the specific patient based on the dosing parameters and patient-specific effective half-life is 56 days, which suggests that the 8 week dosing interval is correct for this specific patient when using the labeled dosage of 5 mg/kg and a target of 5 μg/mL.

The second example, shown with solid arrows, similarly represents a blood sample being taken on the 56^{th} day after a different patient receives a 5 mg/kg dose, but the laboratory testing measures an infliximab concentration of 3 μg/mL in the blood sample, below the target of 5 μg/mL. Taking similar steps as in the first example, the solid arrows show that the measured concentration corresponds to an infliximab effective half-life of 10 days for the specific patient, suggesting that infliximab more quickly leaves the bloodstream of this patient than the patient in the first example. As shown by the solid arrows, the 10 day effective half-life for this patient corresponds to a time to target between 42 and 49 days. Accordingly, a physician may change the dosing regimen for this patient to 5 mg/kg every 6 weeks, instead of every 8 weeks, to account for the patient's lower effective half-life. After administering the new dosing regimen, the physician may use a nomogram with a 42 day concentration curve (not shown) to evaluate the patient's results, for example, seeing a 5 μg/mL measured concentration after 42 days which would suggest the 6 week dosing interval is correct for the patient. Alternatively the physician could increase the administered dosage to account for patient's lower effective half-life demonstrated in the second example in FIG. 1B. The systems described herein may provide time-to-target for different doses (e.g., an increased dose) within one run and output nomograms or time-to-target values for each dose.

The nomogram can be plotted with a region indicating the range of drug effective half-lives of patients who participated in the clinical trials for the drug. If present, the region may be shaded or bounded by a box. Since the clinical trials were used for determining the labeled dosage of the drug, it is helpful for physicians to visualize the variation in effective half-lives beyond those represented by the labeled dosage. For example, effective half-lives for infliximab in clinical trials ranged from about 7.8 days to about 9.5 days. The nomogram results may also be compared to the label regimen—for example, the time-to-target value output for a specific patient is compared to the label interval to show if the label regimen is inappropriate for the specific patient.

While the examples shown in FIGS. 1A and 1B do not account for patient weight, it should be understood that the systems and methods described herein can be utilized to include weight in the PK relationships. For example, the patient's exact weight may be used to create a custom nomogram. Alternatively, the physician may select a nomogram for the patient from a group of nomograms based on different weight classes (e.g., a nomogram for each of a low weight class, a middle weight class, and a high weight class). The nomogram may also account for route of administration. For some drugs, the route affects the pharmacokinetics (and thus the half-life) of the drug, so the nomogram should be constructed for the specific route of administration used by the patient. For example, subcutaneous administration is typically associated with lower bioavailability (compared with intravenous administration), resulting in a higher apparent clearance rate. The user may be able to select from routes including but not limited to subcutaneous, intravenous, oral, intramuscular, intrathecal, sublingual, buccal, rectal, vaginal, ocular, nasal, inhalation, nebulization, cutaneous, or transdermal, and the pharmacokinetic modeling may be adjusted to account for differences between routes.

It should be further understood that FIGS. 1A and 1B represent a specific implementation of the methods described herein for nomogram construction and usage, applied to the drug infliximab. As mentioned previously, nomograms can be constructed for a class of drugs, such as drugs having similar characteristics. For example, a nomogram similar to that in FIG. 1A can be constructed for dosing of monoclonal antibodies.

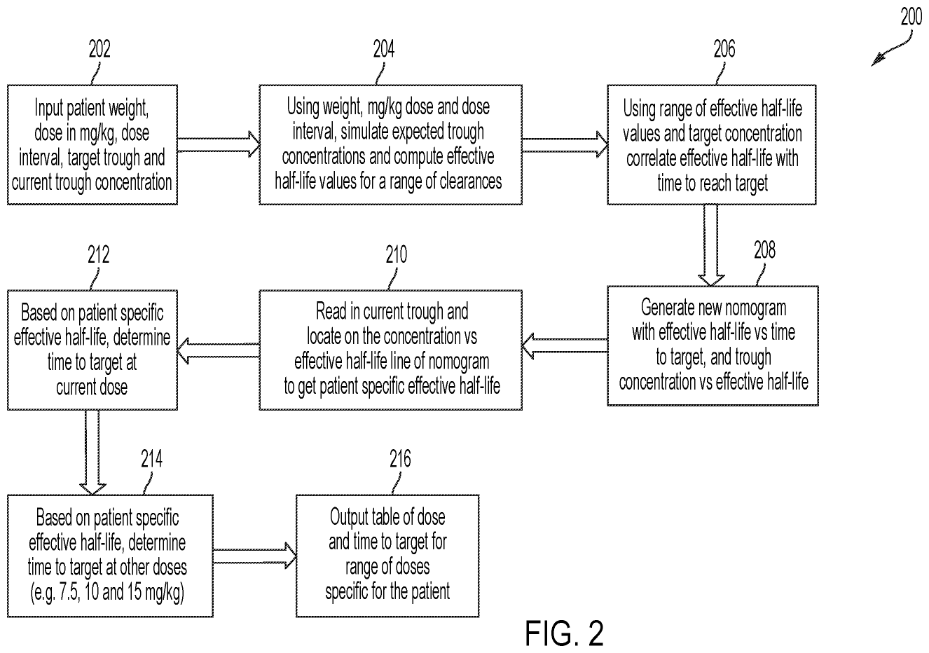

FIG. 2 shows a flowchart describing a method 200 for constructing and using a dosing nomogram for a specific patient. The nomogram is particularly useful for adjusting a dosing regimen (e.g., at least one of the dose amount or the dosing interval). The nomogram may be used for adjusting a dosing regimen for a monoclonal antibody drug. The nomogram may be specific to a drug or a set of drugs. Method 200 comprises steps 202, 204, 206, 208, 210, 212, and, optionally, 214 and 216.

Step 202 involves receiving at an input module data representing the following parameters: a specific patient's weight, an administered dose amount of drug, a current dose interval, a target drug trough concentration, and a measured drug trough concentration in the specific patient. Step 204 involves using the patient weight, the dose amount, and the dose interval to simulate expected trough concentrations and computing the effective half-life values for a range of drug clearance values. Step 206 involves using the range of effective half-life values and the target concentration to simulate time to target values for the effective half-life values. Step 208 involves generating a nomogram by plotting the simulated expected trough concentrations and the simulated time to target values against the effective half-life values. Step 210 involves reading the measured trough concentration on the concentration vs. half-life curve to determine the patient-specific effective half-life. Step 212 involves determining the time to target for the current dose amount based on the patient-specific effective half-life. Optional step 214 involves determining time to target values for other dose amounts based on the patient-specific effective half-life. Optional step 216 involves outputting a table of dose amounts and time to target values for the range of dose amounts for the specific patient. Additional outputs that can be produced by the method include but are not limited to a recommended dosing interval and a recommended dose amount, each of which may be determined based on the time-to-target value(s) for the administered dose amount and target concentration. Method 200 may be implemented using a processor configured with the input module. The steps of method 200 may be embodied in computer-readable medium (e.g., code as computer-readable instructions) for execution by a processor. A graphical user interface may be used to accept the inputted data and display the results of method 200 (including the nomogram and recommended dosing regimens). Method 200 may be used as part of a method of treatment using the drug or set of drugs.

As described above, expected concentrations and time to target values can be simulated over a range of effective half-life values according the pharmacokinetic relationships described by Eqns. 1 and 2, using a pharmacokinetic (at least in part) model. A pharmacokinetic (PK) model can be used to simulate these values for steps 204 and 206. The open two-compartment PK model shown in FIG. 5 and described herein is an example of a model that may be used. A linear clearance relationship and a linear first-order absorption relationship may be used in the model. A PK model can be used by inputting into the model the current dose amount, the current dose interval, and the patient weight. A range of drug clearance values, representing the retention of drug within the body for a population of patients, can be incrementally stepped through in the model to provide a plurality of expected drug trough concentrations. The model is then used to compute a plurality of effective drug half-lives for the given patient weight. Each effective drug half-life correspond to a drug clearance value in the range of clearance values. The model then outputs the plurality of effective drug half-lives as the effective drug half-life range and the plurality of drug trough concentrations as the range of expected drug trough concentrations. Each drug trough concentration corresponds to an effective half-life of the plurality of effective drug half-lives.

In some implementations, the drug is one or more of infliximab, adalimumab, vedolizumab, golimumab, ustekinumab, abatacept, rituximab, ixekizumab, certolizumab pegol, entanercept, dupilumab, tocilizumab, alemtuzumab, secukinumab, guselkumab, reslizumab, mepolizumab, omalizumab, benralizumab, sarilumab, risankizumab, tildrakizumab, ocrelizumab, olokizumab, and natalizumab. A generalized PK model may be used to create a nomogram that applies to a plurality of these drugs. In some implementations, the effective drug half-life range comprises effective half-life values between 2 days and 25 days. When the drug is infliximab, the prior dose amount may be about 5 mg/kg, and the target may be between 1 μg/mL and 20 μg/mL.

As discussed above, the nomogram may be particularly useful for patients undergoing maintenance dosing after completing induction dosing. The patient's effective half-life for the drug may be more stable during the maintenance period, so the nomogram would provide more accurate results. The nomogram may also be used in situations where a physician or user needs to know when the patient's body will be entirely clear of the drug (i.e., setting the target concentration to zero), and the method involves outputting a time-to-target when the patient's body will be entirely clear. This may be useful for determining when patients may be eligible to switch to a new drug or start a clinical trial.

Table 2 below is an example of the table that would be output during step 216 of method 200. The example in Table 2 is based on similar parameters as used for creating the nomogram of FIG. 1A (i.e., 5 μg/mL target concentration, 3 μg/mL measured concentration at 14 weeks, 180 lb patient, prior dose of 5 mg/kg given every 8 weeks). The table includes a plurality of new dose amounts, including 5 mg/kg, 7.5 mg/kg, 10 mg/kg, and 15 mg/kg. The results of method 200 are shown for each new dose amount. It should be understood that Table 2 includes a one specific patient's effective half-life values, i.e., the patient-specific effective half-life determined in method 200 for each of the new doses. The table created in step 216 may be refined to only show rows including the patient-specific effective half-life.

TABLE 2
Infliximab dosing nomogram in tabular form, based on a 5 μg/mL
target concentration, measured concentration of 3 μg/mL at 14 weeks,
70 kg patient, prior dose of 5 mg/kg given every 8 weeks. New dose,
weight, effective half-life, and time to target are tabulated.

| New | | Effective | Days |
|---|---|---|---|
| Dose | Weight | half-life | to |
| (mg/kg) | (kg) | (days) | Target |
| 5.0 | 180 | 10.01 | 39.7 |
| 7.5 | 180 | 10.01 | 47.2 |
| 10.0 | 180 | 10.01 | 52.9 |
| 15.0 | 180 | 10.01 | 61.4 |

The nomogram can also be generated to determine a “washout period” for a patient. According to the U.S. National Library of Medicine, a washout period is defined as “a period of time during a clinical study when a participant is taken off a study drug or other medication in order to eliminate the effects of the treatment.” Washout periods are an important clinical tool for studying the post-treatment effects for patients and also for withdrawing patients from a current treatment before an active treatment begins. Clinicians want to ensure that a patient's body is free from the effects of a previous treatment before starting a new one, in order to minimize cross-effects between the treatments or, in the case of clinical studies, to eliminate effects of the previous treatment so as to get a clearer understanding of the effects of the new treatment. Washout period can be used when a patient fails therapy with a certain drug and plans to start a new drug but needs to go through washout of the failed drug before starting therapy with the new drug. Typically, clinicians have all patients wait a period of 30 days, but each individual patient has a unique effective half-life for a given drug, so the true washout period may be shorter or longer than 30 days. Using a washout nomogram, the clinician can find a more exact washout period for the individual. For example, a patient, with an effective half-life that corresponds to a washout period less than 30 days, would otherwise regress when using the 30 day standard-of-care. Knowing the washout period can also help accelerate drug trails by putting patients on a new drug faster if the patients have a faster than average half-life for a previously administered drug.

A washout period can be estimated as the time it takes for the concentration of a previous drug to reach a washout threshold concentration in the patient's body. For example, the washout threshold may be zero or near zero (e.g., about 0.1, about 0.2, about 0.3, about 0.5, about 1 concentration units, such as μg/mL). The washout period for a patient can be calculated using the same nomogram produced via method 200 by inputting the washout threshold as the target dose at step 202. Thus, a washout nomogram can be constructed by method 200, allowing a clinician to determine when a patient will be free of drug effects. Table 3 below shows example results of outputting the nomogram for determining washout period for a patient on infliximab. As an alternative, rather than producing the washout nomogram by inputting the washout threshold concentration as the target at step 202, the washout nomogram may be constructed as an additional output during a further step of method 200. The user may be able to select an option to produce the washout nomogram after constructing the desired nomogram.

TABLE 3
Infliximab washout nomogram in tabular form, based on a
0.01 μg/mL target concentration (as a washout threshold
concentration for the new dose), measured concentration of
1 μg/mL at 14 weeks, 50 kg patient, prior dose of 5 mg/kg
given every 6 weeks. New dose, weight, effective half-life,
and time to target are tabulated, wherein the time to target is
the washout period for the new dose.

| New | | Effective | Days |
|---|---|---|---|
| Dose | Weight | half-life | to |
| (mg/kg) | (kg) | (days) | Target |
| 5.0 | 50 | 6.0676 | 138.8 |
| 7.5 | 50 | 6.0676 | 146.4 |
| 10.0 | 50 | 6.0676 | 152.1 |
| 15.0 | 50 | 6.0676 | 160.3 |

The results may be stored in a library, such as a memory device or cloud memory architecture. The library may store dose, weight, measured concentration, or any other parameters discussed herein, for each individual patient for whom a nomogram is generated. When another patient with one or more matching parameters is in need of a nomogram, the previously generated nomogram results can be looked up, rather than re-computing the nomogram process, thus saving time and computing efficiency.

A nomogram can be implemented in a graphical user interface comprising the nomogram constructed according to method 200; a plurality of input boxes operatively coupled to the input module of the processor for receiving the data in step 202; a plurality of arrows, lines, or markers (e.g., circles, dots, stars, symbols) on the nomogram indicating the measured drug concentration, the effective drug half-life for the specific patient; and time to target value for the specific patient and the dose amount (or a plurality of time to target values for the specific patient over a range of dose amounts); and an output for displaying the time to target value (or the plurality of time to target values). The interface may include a button or option for producing probability plots of TTFADA plots, as discussed below.

A computer processor or a physician may perform a method for determining a dose interval for the drug for the specific patient by performing method 200 and additionally setting the time to target value (or the plurality of time to target values) to a new dose interval for the dose amount (or for each of the plurality of available dose amounts) of the drug for the specific patient. If the new dose interval is less than a standard of care dose interval, then the method may further comprise providing the patient with a recommendation to use Bayesian individualized dosing, for example, using the systems or methods described in U.S. patent application Ser. No. 15/094,379, entitled “SYSTEMS AND METHODS FOR PATIENT-SPECIFIC DOSING”, filed on Apr. 8, 2016, and published as Publication No. US 2016/0300037, which is hereby incorporated by reference in its entirety.

A physician may perform a method of treatment by administration of the drug using a new dosing regimen determined according to method 200. For example, the new dosing regimen includes a new dose selected from the plurality of available doses, or the dosing regimen includes a dosing interval selected from the time to target values. The method of treatment involves administration of the drug using the new dose interval based on the time to target value(s) discussed above. For example, the method may involve treating any one of inflammatory bowel disease (IBD), rheumatoid arthritis (RA), juvenile idiopathic arthritis (JIA), ankylosing spondylitis (AS), psoriasis (PsO), psoriatic arthritis (PsA), multiple sclerosis (MS), atopic dermatitis, eczema, asthma, or any other suitable condition or disease. For treatment of these conditions, the drugs may be an antibody, a monoclonal antibody, an antibody construct, or a monoclonal antibody construct. Drugs may be administered using the standard-of-care procedures, such as intravenous or subcutaneous administration.

Method 200 may also be applied as a method of rationing drug doses by setting the dose regimen of a drug for a specific patient such that the lowest amount of drug or least frequent interval is used to maintain the target concentration based on the patient-specific effective half-life.

FIGS. 3A-3C show nomograms that are examples of nomograms that would be produced by method 200 of FIG. 2. Similar to the nomogram of FIGS. 1A and 1B, FIGS. 3A-3C are nomograms for dosing of infliximab. The parameters for each nomogram are a 5 μg/mL target concentration, a prior dose amount of 5 mg/kg every 8 weeks, and a new dose amount of 5 mg/kg. These example nomograms are constructed for the 56^{th} day after the beginning of maintenance on week 6 (i.e., the 14^{th} week of treatment, 8 weeks after the prior dose of 5 mg/kg). Each nomogram of FIGS. 3A-3C corresponds to a different patient weight. FIG. 3A depicts the nomogram for a 50 kg patient, FIG. 3B depicts the nomogram for a 70 kg patient, and FIG. 3C depicts the nomogram for a 90 kg patient.

By comparing each nomogram of FIGS. 3A-3C, it is shown that the concentration vs. half-life curve moves upward as the patient weight increases, suggesting that a given measured concentration corresponds to a shorter effective half-life in heavier patients, as one would expect because dose amounts are administered on a per weight basis.

### Systems and Devices

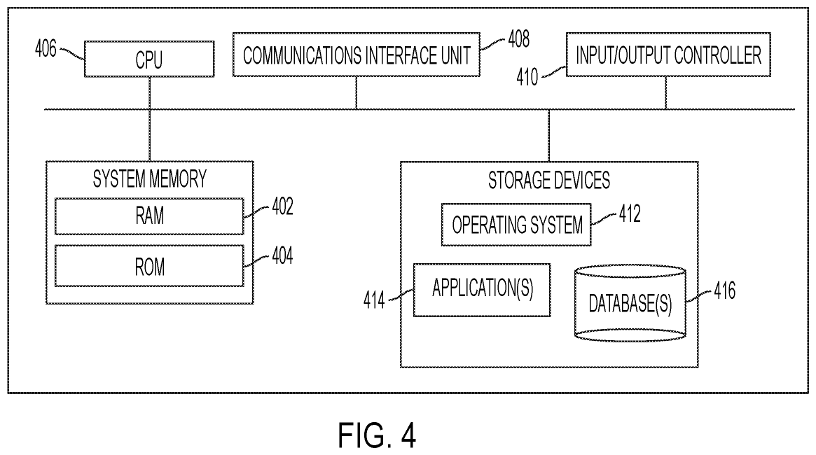

FIG. 4 is a block diagram of a computing device for performing any of the processes described herein. Each of the components of these systems may be implemented on one or more computing devices 400. In certain aspects, a plurality of the components of these systems may be included within one computing device 400. In certain implementations, a component and a storage device may be implemented across several computing devices 400.

The computing device 400 includes at least one communications interface unit, an input/output controller 410, system memory, and one or more data storage devices. The system memory includes at least one random access memory (RAM 402) and at least one read-only memory (ROM 404). All of these elements are in communication with a central processing unit (CPU 406) to facilitate the operation of the computing device 400. The computing device 400 may be configured in many different ways. For example, the computing device 400 may be a conventional standalone computer or alternatively, the functions of computing device 400 may be distributed across multiple computer systems and architectures. In FIG. 4, the computing device 400 is linked, via network or local network, to other servers or systems.

The computing device 400 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 408 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 406 includes a processor, such as one or more conventional microprocessors and one or more supplementary co-processors such as math co-processors for offloading workload from the CPU 406. The CPU 406 is in communication with the communications interface unit 408 and the input/output controller 410, through which the CPU 406 communicates with other devices such as other servers, user terminals, or devices. The communications interface unit 408 and the input/output controller 410 may include multiple communication channels for simultaneous communication with, for example, other processors, servers or client terminals.

The CPU 406 is also in communication with the data storage device. The data storage device may include an appropriate combination of magnetic, optical or semiconductor memory, and may include, for example, RAM 402, ROM 404, flash drive, an optical disc such as a compact disc or a hard disk or drive. The CPU 406 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 406 may be connected to the data storage device via the communications interface unit 408. The CPU 406 may be configured to perform one or more particular processing functions.

The data storage device may store, for example, (i) an operating system 412 for the computing device 400; (ii) one or more applications 414 (e.g., computer program code or a computer program product) adapted to direct the CPU 406 in accordance with the systems and methods described here, and particularly in accordance with the processes described in detail with regard to the CPU 406; or (iii) database(s) 416 adapted to store information that may be utilized to store information required by the program.

The operating system 412 and applications 414 may be stored, for example, in a compressed, an uncompiled and an encrypted format, and may include computer program code. The instructions of the program may be read into a main memory of the processor from a computer-readable medium other than the data storage device, such as from the ROM 404 or from the RAM 402. While execution of sequences of instructions in the program causes the CPU 406 to perform the process steps described herein, hard-wired circuitry may be used in place of, or in combination with, software instructions for implementation of the processes of the present invention. Thus, the systems and methods described are not limited to any specific combination of hardware and software.

Suitable computer program code may be provided for performing one or more functions described herein. The program also may include program elements such as an operating system 412, a database management system and “device drivers” that allow the processor to interface with computer peripheral devices (e.g., a video display, a keyboard, a computer mouse, etc.) via the input/output controller 410.

The term “computer-readable medium” as used herein refers to any non-transitory medium that provides or participates in providing instructions to the processor of the computing device 400 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 406 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 200 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

FIG. 5 depicts an example of a pharmacokinetic (PK) model 500A/500B that may be used to compute the nomograms described herein. 500A shows the model with rate constants k, k₁₂, and k₂₁, while 500B shows the model with simplified parameters Q (intercompartmental clearance) and CL (clearance). This example PK model is a two-compartment model, including the central compartment 504 and peripheral compartment 506. The central compartment 504 may generally represent blood circulation in an organism and corresponds to a relatively rapid distribution. For example, the central compartment may represent organs and systems within an organism that have a well-developed blood supply, such as the liver or kidney or may be restricted to the circulatory system. In contrast, the peripheral compartment 506 may represent organs or systems that have lower blood flow, such as muscle, lean tissue, and fat or may refer to tissues in general as opposed to blood.

In addition to the two compartments in the PK model 500A/500B, FIG. 5 also depicts input flows and output flows into and out of the compartments. In particular, the infusion (not shown) corresponds to a flow rate of entrance of the drug into the body via the site of administration and into the central compartment 504. The clearance (CL) 510 corresponds to the clearance of the central compartment 504, and may be representative of an amount of drug that is flushed out of the system, such as via metabolism or excretion processes. Clearance CL is used to derive the exit rate constant parameter k, where k=CL/V₁. The intercompartmental clearance (Q) 508 corresponds to a distributional clearance between the central compartment 504 and the peripheral compartment 506, and broadly represents distribution of the drug between the blood flow and tissues comprising organs and other body components with lower blood flow. The intercompartmental clearance Q is used to derive rate parameters k₁₂ and k₂₁, representing the flowrate in each direction between compartments 1 and 2. Parameter V₁ corresponds to the volume of distribution of the central compartment 504, and parameter V2 corresponds to the volume of distribution of the peripheral compartment 506. Equations 3 and 4 describe the pharmacokinetic relationships, in the central and peripheral compartments, respectively, of the two-compartment PK model, where [A] is the drug concentration in each compartment 1 and 2:

$$\frac{{d [ A ]}_{1}}{dt} = - \frac{Q}{{V}_{1}} * {[ A ]}_{1} + \frac{Q}{{V}_{2}} * {[ A ]}_{2} - \frac{CL}{{V}_{1}} * {[ A ]}_{1} \qquad Eqn . 3$$

$$\frac{{d [ A ]}_{2}}{dt} = \frac{Q}{{V}_{1}} * {[ A ]}_{1} - \frac{Q}{{V}_{2}} * {[ A ]}_{2} \qquad Eqn . 4$$

Pharmacokinetic and pharmacodynamic models that may be used herein are also described in U.S. patent application Ser. No. 15/094,379, entitled “SYSTEMS AND METHODS FOR PATIENT-SPECIFIC DOSING”, filed on Apr. 8, 2016, and published as Publication No. US 2016/0300037; U.S. patent application Ser. No. 16/391,950, entitled “SYSTEMS AND METHODS FOR MODIFYING ADAPTIVE DOSING REGIMENS”, filed on Apr. 23, 2019, and published as Publication No. US 2019/0326002; and U.S. patent application Ser. No. 16/813,366, entitled “SYSTEMS AND METHODS FOR DRUG-AGNOSTIC PATIENT-SPECIFIC DOSING REGIMENS”, filed on [Mar. 9, 2020], and published as Publication No. [US 2020/0321096], each of which is hereby incorporated by reference in its entirety.

FIG. 6 is a block diagram of a computerized system 600 for implementing the systems and methods disclosed herein. In particular, the system 600 uses medication-specific mathematical models and observed patient-specific responses to treatment to predict, propose, and evaluate suitable medication treatment plans for a specific patient. The system 600 includes a server 604, a clinical portal 614, a pharmacy portal 624, and an electronic database 106, all connected over a network 602. The server 604 includes a processor 605, the clinical portal 614 includes a processor 610 and a user interface 612, and the pharmacy portal 624 includes a processor 620 and a user interface 622. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data that is currently being processed. An illustrative computing device 400, which may be used to implement any of the processors and servers described herein, is described in detail below with reference to FIG. 4. As used herein, “user interface” includes, without limitation, any suitable combination of one or more input devices (e.g., keypads, touch screens, trackballs, voice recognition systems, etc.) and/or one or more output devices (e.g., visual displays, speakers, tactile displays, printing devices, etc.). As used herein, “portal” includes, without limitation, any suitable combination of one or more devices configured with hardware, firmware, and software to carry out one or more of the computerized techniques described herein. Examples of user devices that may implemental a portal include, without limitation, personal computers, laptops, and mobile devices (such as smartphones, blackberries, PDAs, tablet computers, etc.). For example, a portal may be implemented over a web browser or a mobile application installed on the user device. Only one server, one clinical portal 614, and one pharmacy portal 624 are shown in FIG. 6 to avoid complicating the drawing; the system 600 can support multiple servers and multiple clinical portals and pharmacy portals.

In FIG. 6, a patient 616 is examined by a medical professional 618, who has access to the clinical portal 614 (e.g., an electronic medical record (EMR) system, such as the APOLLO™ integrated EMR system). The patient may be subject to a disease that has a known progression, and consults the medical professional 618. The medical professional 618 makes measurements from the patient 616 and records these measurements over the clinical portal 614. For example, the medical professional 618 may draw a sample of the blood of the patient 616, and may measure a concentration of a biomarker in the blood sample. In general, the medical professional 618 may make any suitable measurement of the patient 616, including lab results such as concentration measurements from the patient's blood, urine, saliva, or any other liquid or tissue sampled from the patient. The measurement may correspond to observations made by the medical professional 618 of the patient 616, including any symptoms exhibited by the patient 616. For example, the medical professional 618 may perform an examination of the patient gather or measure patient-specific factors such as sex, age, weight, race, disease stage, disease status, prior therapy, other concomitant diseases and/or other demographic and/or laboratory test result information. More specifically, this involves identifying patient characteristics that are reflected as patient factor covariates within the mathematical model that will be used to predict the patient's response to a drug treatment plan. For example, if the model is constructed such that it describes a typical patient response as a function of weight and gender covariates, the patient's weight and gender characteristics would be identified. Any other characteristics may be identified that are shown to be predictive of response, and thus reflected as patient factor covariates, in the mathematical models. By way of example, such patient factor covariates may include weight, gender, race, lab results, disease stage and other objective and subjective information. Alternatively, the data is automatically transmitted between the clinical portal 614 and the system 600. For example, measured concentration data (or prior dosing data, patient characteristics, or recommended dose amounts) found in EMRs in the clinical portal 614 is transmitted to the system 600 for construction of a nomogram.

Based on the patient's measurement data, the medical professional 618 may make an assessment of the patient's disease status, and may identify a drug suitable for administering to the patient 616 to treat the patient 616. The clinical portal 614 may then transmit the patient's measurements, the patient's disease status (as determined by the medical professional 618), and an identifier of the drug over the network 602 to the server 604, which uses the received data to select one or more appropriate computational models from the models database 606. The appropriate computational models are those that are determined to be capable of predicting the patient's response to the administration of the drug. The one or more selected computational models are used to determine a recommended set of planned dosages of the drug to administer to the patient, and the recommendation is transmitted back over the network 602 to the clinical portal 614 for viewing by the medical professional 618.

Alternatively, the medical professional 618 may not be capable of assessing the patient's disease status or identify a drug, and either or both of these steps may be performed by the server 604. In this case, the server 604 receives the patient's measurement data, and correlates the patient's measurement data with the data of other patients in the patient database 606a. The server 604 may then identify other patients who exhibited similar symptoms or data as the patient 616 and determine the disease states, drugs used, and outcomes for the other patients. Based on the data from the other patients, the server 604 may identify the most common disease states and/or drugs used that resulted in the most favorable outcomes, and provide these results to the clinical portal 614 for the medical professional 618 to consider.

As is shown in FIG. 6, the database 606 includes a set of four databases including a patient database 606a, a disease database 606b, a treatment plan database 606c, and a models database 606d. These databases store respective data regarding patients and their data, diseases, drugs, dosage schedules, and computational models. In particular, the patient database 606a stores measurements taken by or symptoms observed by the medical professional 618. The disease database 606b stores data regarding various diseases and possible symptoms often exhibited by patients infected with a disease. The treatment plan database 606c stores data regarding possible treatment plans, including drugs and dosage schedules for a set of patients. The set of patients may include a population with different characteristics, such as weight, height, age, sex, and race, for example. The models database 606d stores data regarding a set of computational models that may be used to describe PK, PD, or both PK and PD changes to a body. One example of a PK/PD model is described in relation to FIG. 5.

Any suitable mathematical model may be stored in the models database 606d, such as in the form of a compiled library module, for example. In particular, a suitable mathematical model is a mathematical function (or set of functions) that describes the relationship between a dosing regimen and the observed patient exposure and/or observed patient response (collectively “response”) for a specific medication. Accordingly, the mathematical model describes response profiles for a population of patients. Generally, development of a mathematical model involves developing a mathematical function or equation that defines a curve that best “fits” or describes the observed clinical data, as will be appreciated by those skilled in the art. Typical models also describe the expected impact of specific patient characteristics on response, as well as quantify the amount of unexplained variability that cannot be accounted for solely by patient characteristics. In such models, patient characteristics are reflected as patient factor covariates within the mathematical model. Thus, the mathematical model is typically a mathematical function that describes underlying clinical data and the associated variability seen in the patient population. These mathematical functions include terms that describe the variation of an individual patient from the “average” or typical patient, allowing the model to describe or predict a variety of outcomes for a given dose and making the model not only a mathematical function, but also a statistical function, though the models and functions are referred to herein in a generic and non-limiting fashion as “mathematical” models and functions.

It will be appreciated that many suitable mathematical models already exist and are used for purposes such as drug product development. Examples of suitable mathematical models describing response profiles for a population of patients and accounting for patient factor covariates include PK models, PD models, hybrid PK/PD models, and exposure/response models. Such mathematical models are typically published or otherwise obtainable from medication manufacturers, the peer-reviewed literature, and the FDA or other regulatory agencies. Alternatively, suitable mathematical models may be prepared by original research.

Often, the medical professional 618 may be a member or employee of a medical center. The same patient 616 may meet with multiple members of the same medical center in various roles. In this case, the clinical portal 614 may be configured to operate on multiple user devices. The medical center may have its own records for the particular patient. In some implementations, the present disclosure provides an interface between the computational models described herein and a medical center's records. For example, any medical professional 618, such as a doctor or a nurse, may be required to enter authentication information (such as a username and password) or scan an employee badge over the user interface 612 to log into the system provided by the clinical portal 614. Once logged in, each medical professional 618 may have a corresponding set of patient records that the professional is allowed to access.

In some implementations, the patient 616 interacts with the clinical portal 614, which may have a patient-specific page or area for interaction with the patient 616. For example, the clinical portal 614 may be configured to monitor the patient's treatment schedule and send appointments and reminders to the patient 616. Moreover, one or more devices (such as smart mobile devices or sensors) may be used to monitor the patient's ongoing physiological data, and report the physiological data to the clinical portal 614 or directly to the server 604 over the network 602. The physiological data is then compared to expectations, and deviations from expectations are flagged. Monitoring the patient's data on a continual basis in this manner allows for possible early detection of deviations from expectations of the patient's response to a drug, and may indicate the need for early intervention or alternate therapy.

As described herein, the measurements from the patient 616 that are provided into the computational model may be determined from the medical professional 618, directly from devices monitoring the patient 616, or a combination of both. Because the computational model predicts a time progression of the disease and the drug, and their effects on the body, these measurements may be used to update the model parameters, so that the treatment plan (that is provided by the model) is refined and corrected to account for the patient's specific data.

In some implementations, it is desirable to separate a patient's personal information from the patient's measurement data that is needed to run the computational model. In particular, the patient's personal information may be protected health information (PHI), and access to a person's PHI should be limited to authorized users. One way to protect a patient's PHI is to assign each patient to an anonymized code when the patient is registered with the server 604. The code may be manually entered by the medical professional 618 over the clinical portal 614, or may be entered using an automated but secure process (e.g., a secure data vault). The server 604 may be only capable of identifying each patient according to the anonymized code, and may not have access to the patient's PHI. In particular the clinical portal 614 and the server 604 may exchange data regarding the patient 616 without identifying the patient 616 or revealing the patient's PHI.

The generation or selection of the code may be performed in a similar manner as is done for credit card systems. For example, all access to the system may be protected by an application programming interface (API) key. Moreover, when the medical professional 618 is part of a medical center, the medical center's connection to the network 602 over the clinical portal 614 may have enhanced security systems in compliance with HIPAA. As an example, a single administrative database may define access in a manner that ensures that members of one team (e.g., one set of medical professionals, for example) are prohibited from viewing records associated with another team. To implement this, each end-user application may be issued a single API key that specifies which portions of a database may be accessed.

In some implementations, multiple levels of clinician interaction with the portal are configured. For example, some medical professionals, upon logging into the clinical portal 614, may have access that only allows them to view the patient's data. Another level of access may allow the medical professional 618 to view the patient's data as well as enter measurement and observation data regarding the patient 616. A third level of access may allow the medical professional 618 to view and update the patient's data, as well as prescribe a treatment for the patient 616 or otherwise update the patient's treatment plan or dosing schedule.

Different levels of access may be set for different types of users. For example, a user who is a system administrator for the clinical portal 614 may be able to grant or rescind access to the system to other users, but does not have access to any patient records. As another example, a prescriber may be allowed to modify a particular patient's treatment plan and has read and write access to patient records. A reviewer may have just read-only access to patient records, and can only view a patient's treatment plan. A data manager may have read and write access to the patient records, but may not be allowed to modify a patient's treatment plan.

In some implementations, the clinical portal 614 is configured to communicate with the pharmacy portal 624 over the network 602. In particular, after a dosing regimen is selected to be administered to the patient 616, the medical professional 618 may provide an indication of the selected dosing regimen to the clinical portal 614 for transmitting the selected dosing regimen to the pharmacy portal 624. Upon receiving the dosing regimen, the pharmacy portal 624 may display the dosing regimen and an identifier of the medical professional 618 over the user interface 622, which interacts with the pharmacist 628 to fulfill the order.

In some implementations, recommendations or custom orders for drug amounts is provided to drug manufacturers (not shown), who may have access to the network 602. Manufacturers of drugs may only produce certain drugs at set amounts or volumes, which may correspond to recommended dosage amounts for the “typical” patient. This may be especially true for expensive drugs. However, as is described herein, the optimal amount or dosing schedule of a drug for a specific patient may be different for different patients. Moreover, some drugs have expiration dates or have decreased efficacy over time as the drug sits on the shelf. Thus, if it is desirable to administer the optimal amount of drug according to a recommended dosing regimen, then this could potentially lead to drug wastage at least because the optimal amount may not correspond to an integer multiple of the set amount that is produced by the manufacturer.

One way for this problem to be mediated is to provide information to the drug manufacturer reflective of the recommended dosing regimen ahead of time, so that the drug manufacturer can produce custom sized orders for certain medications at the desired times according to the regimen. In this manner, the present disclosure allows for drugs to be freshly produced in the desired amounts at a time that is as close to the administration time as possible.

Moreover, clinical phase IV drug trials are often limited due to the expensive cost of the drugs. The present disclosure provides a way for data regarding a subject's specific response to a drug to be fed back into the models to adequately capture the subject's specific data. The present disclosure provides an automated method of computing a recommended dosage schedule that is deterministic. The dosage schedule can be supplied economically and quickly in a secure manner (e.g., without revealing the patient's PHI) to the drug manufacturer, who may then manufacture customized orders, thereby saving on cost and leading to reduced drug wastage. Moreover, the manufacturer of the drug may be interested in the tested efficacy of the drug, and may be able to adjust the amounts of the drug that are produced and/or the production timeline to accommodate various dosing regimens.

In addition, to the extent that a drug manufacturer's timeline is limited by certain factors, the present disclosure is capable of providing recommended dosing regimens within the limits of the drug manufacturer. For example, for technological and/or economical reasons, the drug manufacturer may only be able to produce a drug in set quantities. Because a dosing regimen often involves two parameters (namely, an amount of a drug and a time at which to administer the drug), the recommended dosing regimen provided by the system 100 may be modified accordingly to accommodate the drug manufacturer's limits.

As is shown in FIG. 6, the server 604 is a device (or set of devices) that is remote from the clinical portal 614. Depending on the computational power of the device that houses the clinical portal 614, the clinical portal 614 may simply be an interface that primarily transfers data between the medical professional 618 and the server 604. Alternatively, the clinical portal 614 may be configured to locally perform any or all of the steps described to be performed by the server 604, including but not limited to receiving patient symptom and measurement data, accessing any of the databases 606, running one or more computational models, and providing a recommendation for a dosage schedule based on the patient's specific symptom and measurement data. Moreover, while FIG. 6 depicts the patient database 606a, the disease database 606b, the treatment plan database 606c, and the models database 606d as being entities that are separate from the server 604, the clinical portal 614, or the pharmacy portal 624, one of ordinary skill in the art will understand that any or all of the databases 606 may be stored locally on any of the devices or portals described herein, without department from the scope of the present disclosure.

### ADDITIONAL IMPLEMENTATIONS

Effective half-life is described in the foregoing as a useful predictor for determining time-to-target for a dosing regimen, but it is also useful for predicting other aspects of the patient's response to treatment. Using a dataset of patient responses for a population of patients, each patient having a clearance and effective half-life, a logistic regression can be applied to the dataset to model selected responses of interest across a range of effective half-lives, similar to the nomogram described in the foregoing. Logistic regression (or logit) models are generally used to model the probability of a certain class or event existing such as pass/fail, win/lose, alive/dead or healthy/sick. This can be extended to model several classes of events such as determining whether a patient is likely to have a certain clinical outcome. Each possible outcome for a given class would be assigned a probability between 0 and 1, with a sum of one. The output of the modeling the selected patient responses is one or more probability plots, which shows the probability of each outcome of a selected response (e.g., on the y-axis) over a range of effective half-lives (e.g., on the x-axis). The range of effective half-lives may be known (e.g., included in the dataset) for the patients of the dataset, or the effective half-life of each patient in the dataset may be estimated based on observed pharmacokinetic data (e.g., by using Eqs. 1 and 2). Examples of patient responses that are relevant (e.g., to IBD patients) and may be modeled this way include but are not limited to Crohn's disease activity index (CDAI), mucosal healing, fecal calprotectin (FCP) (either normalized or non-normalized), C-reactive protein (CRP) concentration, presence or development of anti-drug antibodies (ADA), steroid usage, Mayo score, partial Mayo score, Harvey-Bradshaw index, presence or concentration of Factor VIII protein, and other suitable patient responses or parameters. It should also be understood that composite scores may be generated based on a combination (e.g., weighted or equal combination) of two or more of the probabilities of these responses for individual patients.

The probability plot(s) may be constructed in addition to the nomograms described in the foregoing. For example, a system or user interface, before, during or after generating a dosing nomogram (e.g., by method 200), may allow the user to select an option to produce one or more probability plots, tables, or value outputs based on the effective half-life or effective half-life range used to construct the nomogram. One or more probability plots may be automatically generated by the system or interface. Using the effective half-life estimated for the specific patient based on measured concentration, the probability for the selected response for the individual patient can be read from the plot or simply output as a value. Alternatively, one or more probability plots (or tables) may be generated without a dosing nomogram. The probability plot may be configured with two y-axes, one showing the probabilities for the selected response and the other showing measured drug concentration, such that a curve is generated based on the correlation between measured drug concentration and effective half-life, allowing the user to read the probability from the plot for a given measured drug concentration (and corresponding effective half-life).

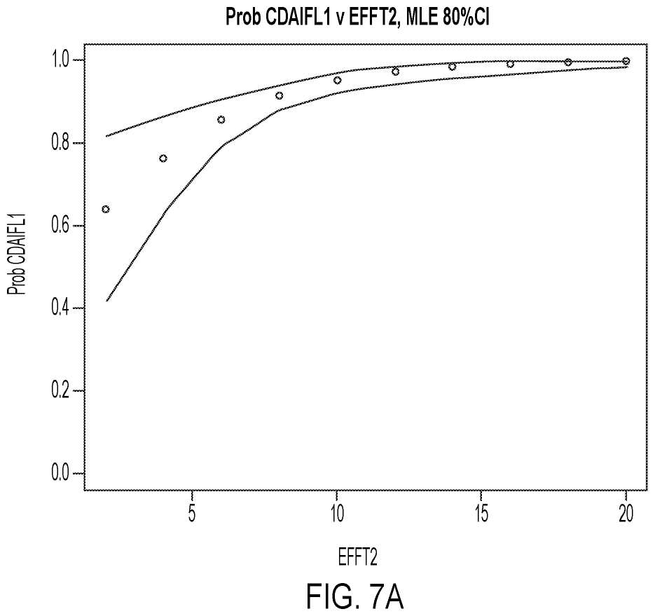

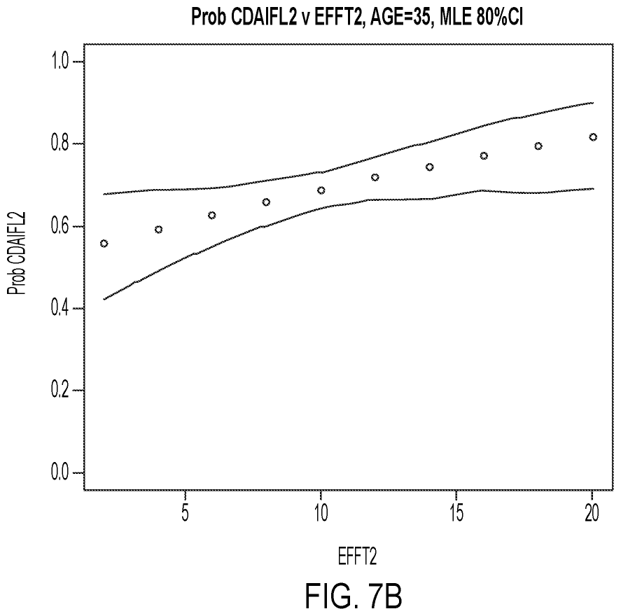

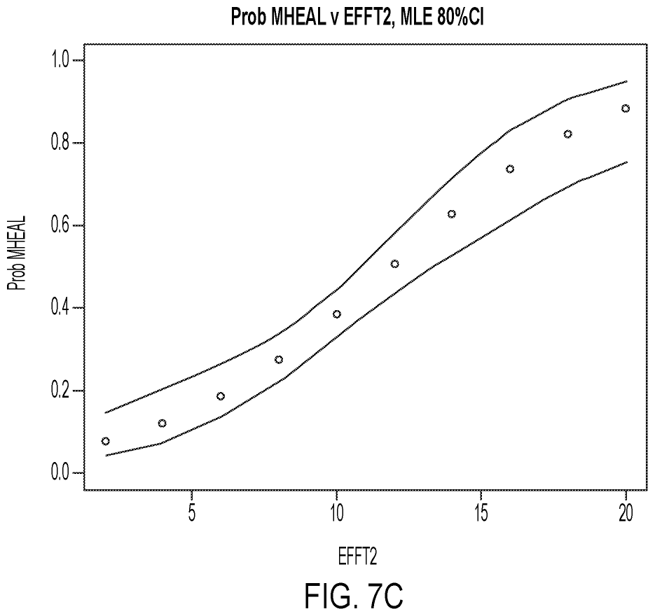

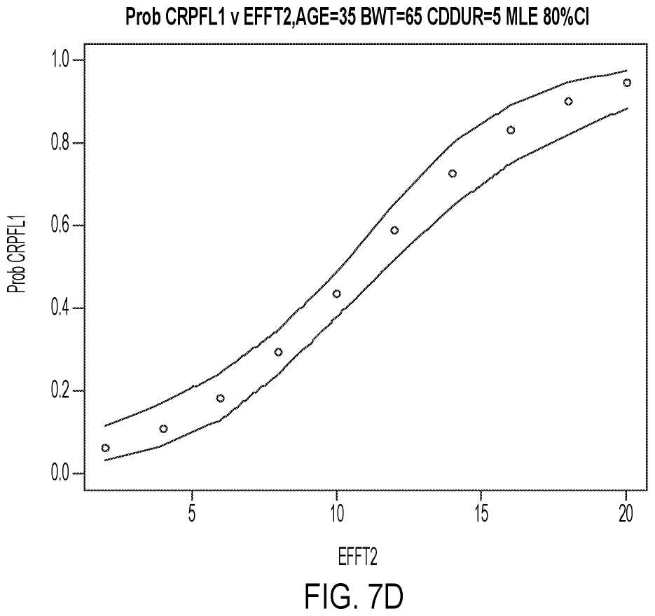

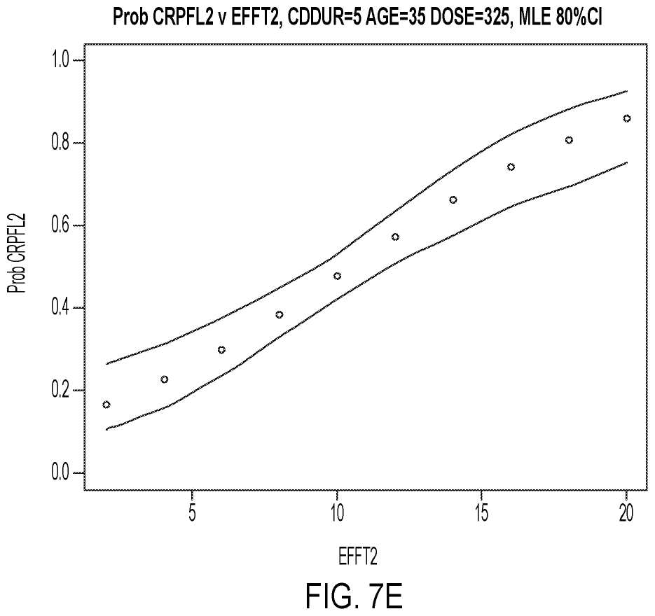

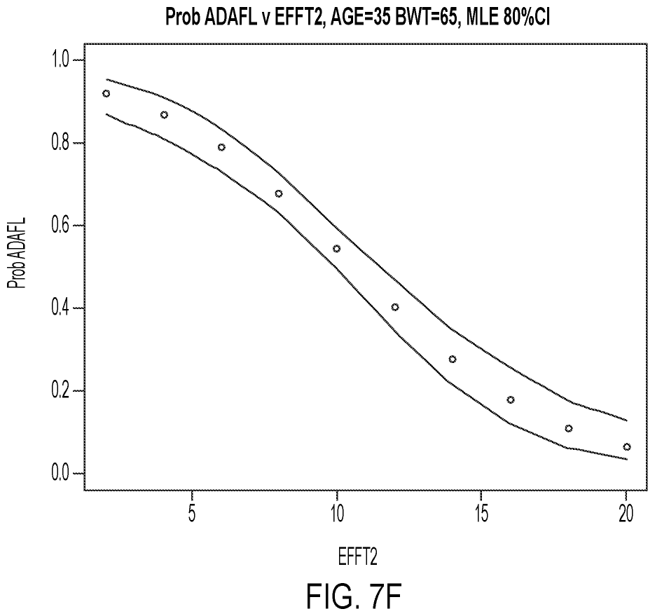

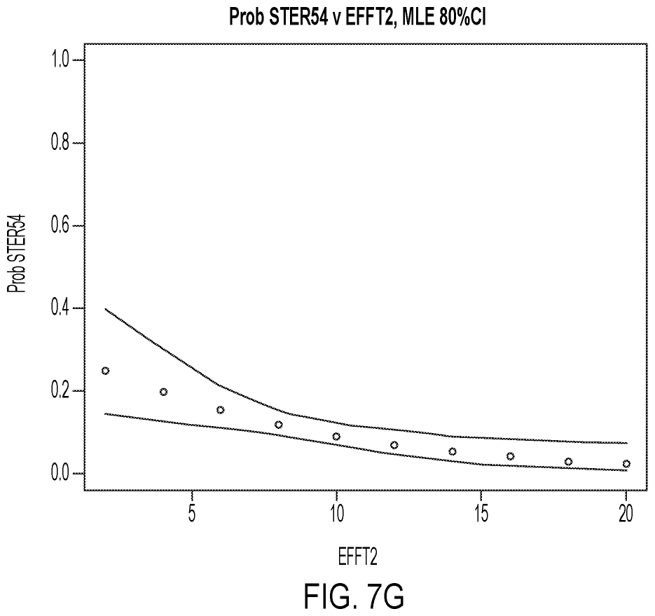

FIGS. 7A-7G show example probability plots of various patient responses of interest against estimated effective half-life. These example probability plots are based on a dataset containing 44 different responses for a population of 220 patients. For selected responses (e.g., those that best predicted by effective half-life), the probability of response versus effective half-life were generated. Although effective half-life is the only predictor used here, it should be understood that other predictors, such as but not limited to baseline age, baseline weight (BWT), dose, Crohn's disease duration (CDDUR), and sex, may be used to generate probability plots of the various responses, along with confidence intervals (which should be understood as an optional step). All possible models using the various predictors may be ranked, e.g. by using Akaike Information Criteria (AIC), and the most parsimonious model (e.g., with the lowest AIC) that contained predictors may be chosen.

FIG. 7A shows the probability of the Crohn's disease activity index (CDAI) at week 30 being 70 points less than baseline, against a range of estimated effective half-lives. The probability is one if the CDAI at week 30 is 70 points less than baseline; otherwise, it is zero. The dataset contained 180 complete cases for this response. The 80% confidence interval is shown by the upper and lower lines bounding the circles which denote the median probabilities. The best model for this response contained only the predictor estimated effective half-life.

FIG. 7B shows the probability of CDAI at week 30 being 150 points less than baseline. The probability is one if the CDAI at week 30 is 150 points less than baseline; otherwise, it is zero. The dataset contained 180 complete cases for this response. The 80% confidence interval is shown by the upper and lower lines bounding the circles which denote the probabilities. The best model for this response contained only the predictors estimated effective half-life and baseline age. Baseline age was fixed at 35 for this example plot.

FIG. 7C shows the probability of mucosal healing evident at final colonoscopy. The probability is one if mucosal healing was evident at final colonoscopy; otherwise, it equals zero. The dataset contained 133 complete cases for this response. The 80% confidence interval is shown by the upper and lower lines bounding the circles which denote the probabilities. The best model for this response contained only the predictor estimated effective half-life.

FIG. 7D shows the probability of C-reactive protein (CRP) concentration in normal range (less than 10 mg/L) at week 30. The probability is one if C-reactive protein (CRP) concentration is in normal range (less than 10 mg/L) at week 30; otherwise, it equals zero. The dataset contained 180 complete cases for this response. The 80% confidence interval is shown by the upper and lower lines bounding the circles which denote the probabilities. The best model for this response contained only the predictors estimated effective half-life, baseline age, BWT, and CDDUR. For this example plot, baseline age was fixed at 35, BWT was fixed at 65 kg, and CDDUR was fixed at 5 years.

FIG. 7E shows the probability of CRP concentration in normal range (less than 10 mg/L) at week 54. The probability is one if CRP concentration is in normal range (less than 10 mg/L) at week 54; otherwise, it equals zero. The dataset contained 170 complete cases for this response. The 80% confidence interval is shown by the upper and lower lines bounding the circles which denote the probabilities. The best model for this response contained only the predictors estimated effective half-life, baseline age, dose, and CDDUR. For this example plot, baseline age was fixed at 35, dose was fixed at 325, and CDDUR was fixed at 5 years.

FIG. 7F shows the probability of anti-drug antibody (ADA) development. The probability is one if ADA were developed; otherwise, it equals zero. The 80% confidence interval is shown by the upper and lower lines bounding the circles which denote the probabilities. The best model for this response contained only the predictors estimated effective half-life, baseline age, and BWT. For this example plot, baseline age was fixed at 35 and BWT was fixed at 65 kg.

FIG. 7G shows the probability of steroid usage at week 54. The probability is one if steroids were used at week 54 (ignoring steroid usage prior to the study drug); otherwise, it equals zero. The 80% confidence interval is shown by the upper and lower lines bounding the circles which denote the probabilities. The best model for this response contained only the predictor estimated effective half-life.

Another usage of the effective half-life approach to estimating patient response is time-to-event analysis for evaluation of the time to first appearance of anti-drug antibody (TTFADA) after administration of a drug to the patient. ADAs are produced by the body's immune response to an administered drug, and ADAs can inactivate the effects of the drug treatment and in some cases induce adverse effects on the patient. Thus, it is useful for clinicians to understand not only the risk that the patient may develop ADAs but also an estimate of when the ADA development may begin. Using population data and a logistic regression, similar as described above for the probability plots, TTFADA can be estimated across a range of effective half-lives (either known for each patient in the population or estimated based on observed pharmacokinetics).

ADA data can be described as interval or right-censored for subjects experiencing or never experiencing positive ADA titers, respectively. Subjects in the population with ADA present at baseline may be discarded from the analysis. Those who develop ADA which subsequently disappears and then re-appears may be only assessed up to the TTFADA. Initially, a dataset may be used for an graphical, non-parametric evaluation for each predictor in the dataset. These can be the same predictors discussed above in relation to FIGS. 7A-7G. Example predictors include but are not limited to baseline age, baseline weight (BWT), estimated effective half-life, Crohn's disease duration (CDDUR), dose, sex, and presence of immune-modulators (IMM) (e.g., azathioprine (AZA) or methotrexate (MTX)). Continuous predictors can be trichotomized into lower quartile, inter-quartile range, and upper quartile bins, so that evidence of hormesis (a dose response phenomenon characterized by a low dose stimulation, zero dose and high dose inhibition thus resulting in a J-shaped or an inverted U-shaped dose response) can be identified. Kaplan-Meier survivor estimates (KM) can be plotted by bin. To construct the TTFADA values for these plots, the following parameters can be used from each patient in the population: day of last negative ADA result, day of first positive ADA result, and day of last negative ADA result prior to the first positive ADA result.

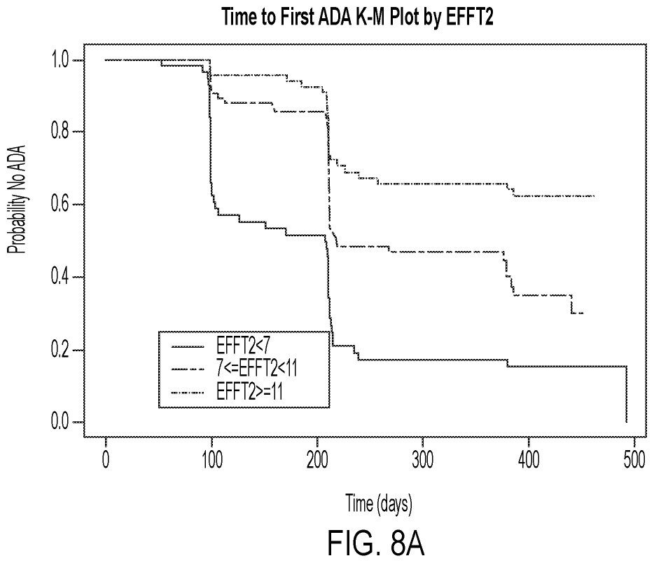

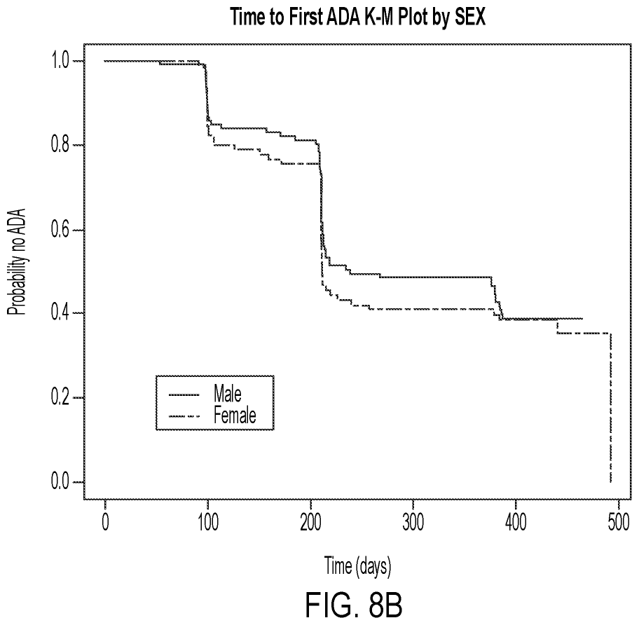

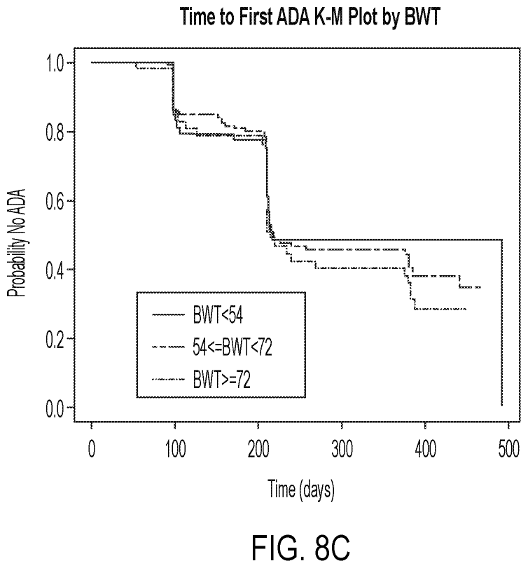

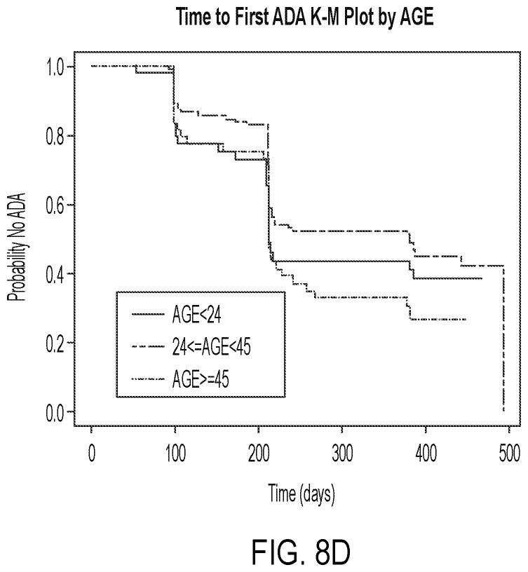

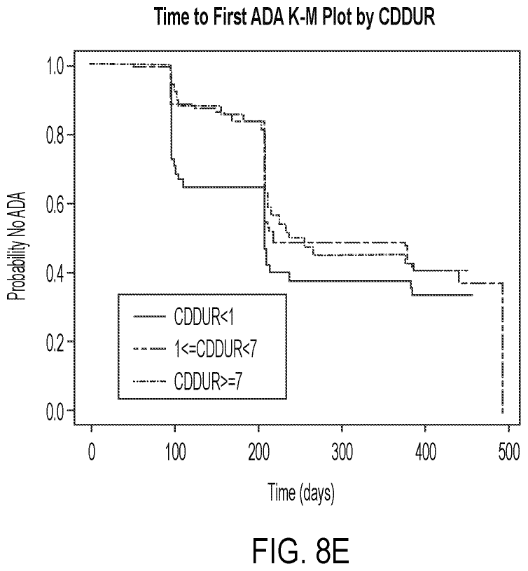

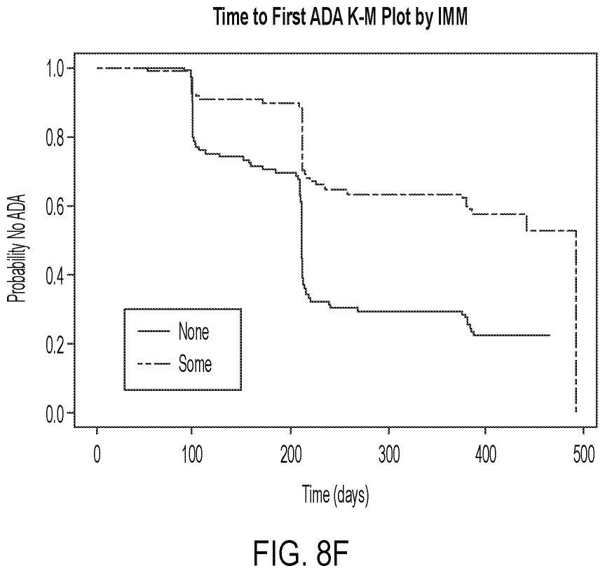

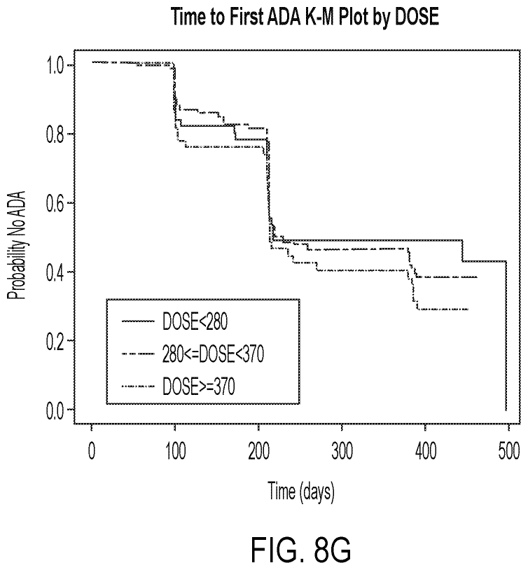

FIGS. 8A-8G show example KM plots for TTFADA against various predictors. These plots were generated according to the above technique, using a clinical dataset containing information on 50 responses or predictors for 220 subjects, each having an effective half-life. Table 4 shows the bin counts for each predictors in this dataset. KM plots are generated for TTFADA against each predictor, stratified across the bins. FIG. 8A shows a TTFADA KM plot by estimated effective half-life. FIG. 8B shows a TTFADA KM plot by sex. FIG. 8C shows a TTFADA KM plot by baseline weight (BWT). FIG. 8D shows a TTFADA KM plot by age. FIG. 8E shows a TTFADA KM plot by Crohn's disease duration (CCDUR). FIG. 8F shows a TTFADA KM plot by presence of immune-modulators (IMM). FIG. 8G shows a TTFADA KM plot by dose.

TABLE 4
Bin counts for the categories of each predictor in
the population dataset.

| Predictor | Category | Count |
|---|---|---|
| Sex | Female | 94 |
| | Male | 119 |
| BWT | <54 kg | 54 |
| | [54 kg, 72 kg) | 106 |
| | ≥72 kg | 53 |
| Age | <24 yrs | 45 |
| | [24 yrs, 45 yrs) | 112 |
| | ≥45 yrs | 55 |
| CDDUR | <1 yrs | 51 |
| | [1 yrs, 7 yrs) | 112 |
| | ≥7 yrs | 50 |
| Est. Eff. | <7 | 57 |
| Half-life | [7, 11) | 97 |
| | ≥11 | 69 |
| IMM | AZA or MTX Present | 98 |
| | Neither | 115 |
| Dose | <280 mg | 55 |
| | [280 mg, 370 mg) | 105 |
| | 370 mg ≤ Dose | 53 |

The resulting data can then be modeled parametrically by constructing a full model. For example, a population pharmacokinetic modeling software can be used to construct the full model. Mixed effects modeling may be used. For example, NONMEM® or a similar software may be used. The constructed full model may then be further refined, e.g., using the Wald's Approximation Method (WAM) algorithm, to select significant predictors. For statistically or clinically significant predictors, hazard ratios (useful for comparing relative hazards) and probability of having an ADA at time points of interest can be calculated. In some implementations, multiple models using different combinations of predictors are output and, optionally, compared to select a final model. Selection of the final model may be based, at least in part, objective function value, Schwarz' Bayesian Criterion (SBC) (e.g., from the modeling software), or approximate SBC (e.g., SBC approximated by WAM).

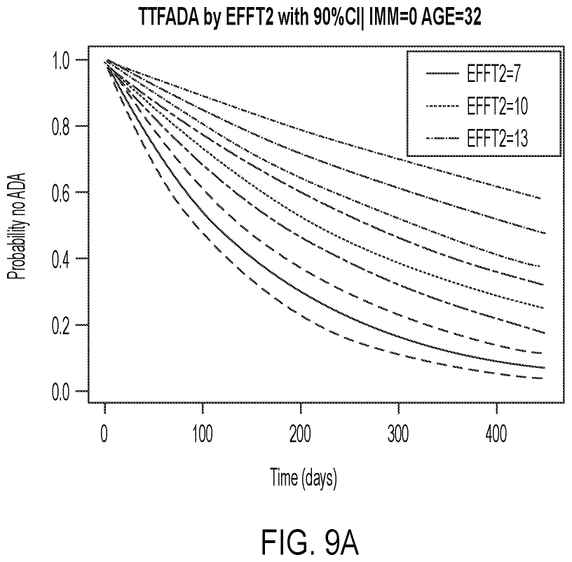

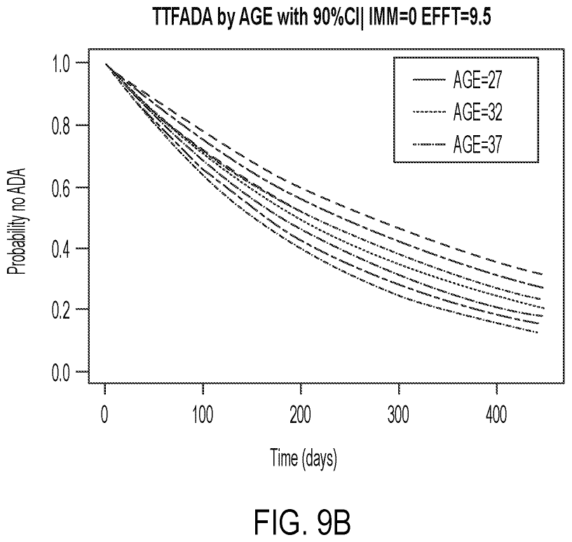

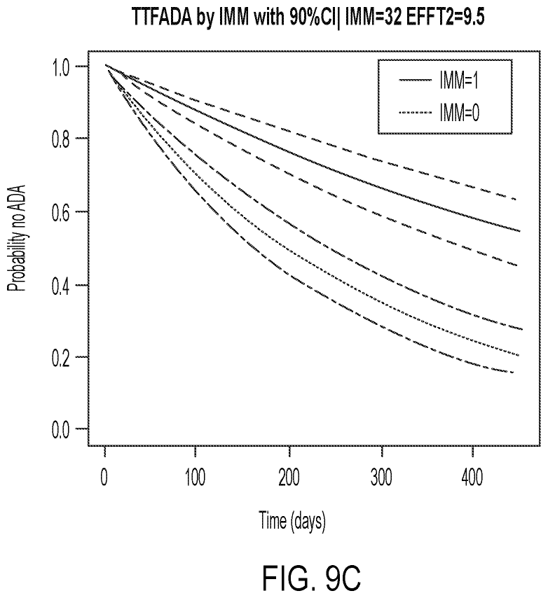

Using the dataset from FIGS. 8A-8G, the full model was constructed and refined. The probabilities of not having an ADA, for the significant predictors (effective half-life, age, and IMM), were plotted over a range of time points to obtain the survivor plots in FIGS. 9A-9C. The probability of one represents absence of ADA, while the probability of zero represents presence of ADA. These plots can be used to estimate the TTFADA for an individual patient or the risk of developing ADA at certain times, based on their individualized values for these predictors. FIG. 9A shows a TTFADA survivor plot with estimated effective half-life bins, with IMM fixed at 0 (absence of IMM) and age fixed at 32. FIG. 9B shows a TTFADA survivor plot with age bins, with IMM fixed at 0 (absence of IMM) and effective half-life fixed at 9.5 days. FIG. 9C shows a TTFADA survivor plot with IMM bins (1 is presence of IMM, 0 is absence of IMM), with age fixed at 32 and effective half-life fixed at 9.5.

Increasing effective half-life was associated with longer TTFADA. Increasing age was associated with shorter TTFADA. Presence of IMM was associated with longer TTFADA. Effective half-life and age cannot be controlled by a caregiver; however, IMM is an external predictor, so use of IMM may be beneficial for delaying ADA development. Effective half-life indication of TTFADA may also be useful for guiding dose adjustment to minimize ADA generation and drug cost.

TTFADA plots (KM or survivor plots) may be included with the nomogram. For example, the patient's predictor values and the range of effective half-lives used to construct a dosing nomogram may be used to produce a TTFADA plot along with the dosing nomogram or as an optional choice after outputting the nomogram. The TTFADA may be used by a clinician to adjust dosing or recommend a different treatment for the patient.

It is to be understood that while various illustrative implementations have been described, the forgoing description is merely illustrative and does not limit the scope of the invention. While several examples have been provided in the present disclosure, it should be understood that the disclosed systems, components and methods of manufacture may be embodied in many other specific forms without departing from the scope of the present disclosure.

The examples disclosed can be implemented in combinations or sub-combinations with one or more other features described herein. A variety of apparatus, systems and methods may be implemented based on the disclosure and still fall within the scope of the invention. Also, the various features described or illustrated above may be combined or integrated in other systems or certain features may be omitted, or not implemented.

While various implementations of the present disclosure have been shown and described herein, it will be obvious to those skilled in the art that such implementations are provided by way of example only. Numerous variations, changes, and substitutions will now occur to those skilled in the art without departing from the disclosure. It should be understood that various alternatives to the implementations of the disclosure described herein may be employed in practicing the disclosure.

All references cited herein are incorporated by reference in their entirety and made part of this application.

1. A method of treating a specific patient with a personalized therapeutic dosing regimen of a drug comprising a monoclonal antibody or monoclonal antibody construct, the method comprising:
receiving at an input module of a processor (1) data indicative of a target drug trough concentration, (2) data indicative of a prior dose amount of the drug, (3) data indicative of a patient's weight of the specific patient, (4) data indicative of a current dose interval, (5) data indicative of a measured drug trough concentration in the specific patient;
simulating an effective drug half-life range and a corresponding range of expected drug trough concentrations at the current dose interval based on the patient's weight, a range of drug clearance values, the current dose interval, and the prior dose amount of the drug;
plotting the corresponding range of expected drug trough concentrations against the effective drug half-life range as a drug concentration curve on a nomogram, useful for adjusting at least one of a dose and a dose interval of a dosing regimen of the drug for administering to the specific patient;
identifying the measured drug trough concentration in the specific patient on the drug concentration curve on the nomogram;
determining an effective drug half-life of the specific patient based on the identified measured drug concentration on the drug concentration curve;
simulating a plurality of time-to-target values for the specific patient based on the determined drug effective half-life and the target drug trough concentration, each time-to-target value corresponding to an available dose in a plurality of available doses; and
administering a new dose of the plurality of available doses of the drug to the specific patient.

2. The method of claim 1, wherein the processor is configured with a pharmacokinetic model, and wherein simulating the effective drug half-life range and corresponding range of expected drug trough concentrations comprises:
inputting into the pharmacokinetic model the prior dose amount, the current dose interval, and the patient weight;
incrementally stepping through a plurality of drug clearance values in the range of drug clearance values, using the pharmacokinetic model, to provide a plurality of expected drug trough concentrations;
computing, using the pharmacokinetic model, a plurality of effective drug half-lives for the patient weight, each effective drug half-life corresponding to a drug clearance value of the plurality of drug clearance values; and
outputting from the pharmacokinetic model the plurality of effective drug half-lives as the effective drug half-life range and the plurality of drug trough concentrations as the range of expected drug trough concentrations, wherein each drug trough concentration corresponds to an effective drug half-life of the plurality of effective drug half-lives.

3. The method of claim 2, wherein the pharmacokinetic model is an open two-compartment model with at least one of a linear clearance and a linear first order absorption.

4. The method of claim 1, wherein the effective drug half-life range comprises effective half-lives between 2 days and 25 days.

5. The method of claim 1, wherein the specific patient is a patient undergoing maintenance dosing and wherein the maintenance dosing begins with a first maintenance dose after an induction dosing period is completed.

6. The method of claim 1, wherein the drug comprises infliximab.

7. The method of claim 6, wherein the prior dose amount comprises 5 mg/kg of infliximab.

8. The method of claim 7, wherein the target concentration is between 1 μg/mL and 20 μg/mL.

9. The method of claim 1, wherein the drug comprises any one of adalimumab, vedolizumab, golimumab, ustekinumab, abatacept, rituximab, ixekizumab, certolizumab pegol, entanercept, dupilumab, tocilizumab, alemtuzumab, secukinumab, guselkumab, reslizumab, mepolizumab, omalizumab, benralizumab, sarilumab, risankizumab, tildrakizumab, ocrelizumab, and natalizumab.

10. The method of claim 1, further comprising determining a label dosage for the drug by plotting a region of effective drug half-lives of patients who participated in clinical trials for the drug.

11. The method of claim 1, further comprising generating a probability plot of a probabilities of a patient response over the effective drug half-life range, wherein the probabilities are determined using a logistical regression of a dataset for a patient population and the dataset comprises a patient response for each patient in the population.

12. The method of claim 11, wherein the dataset further comprises an effective drug half-life for each patient in the population.

13. The method of claim 12, wherein the patient response is one of: Crohn's disease activity index (CDAI), mucosal healing, fecal calprotectin (FCP) concentration, C-reactive protein (CRP) concentration, development of anti-drug antibodies (ADA), steroid usage, Mayo score, partial Mayo score, Harvey-Bradshaw index, and concentration of Factor VIII protein.

14. The method of claim 1, further comprising generating a plot of probabilities of anti-drug antibody presence over time, wherein a probability-time curve is generated for each of a set of effective drug half-life sub-ranges.

15. The method of claim 14, further comprising evaluating a time-to-first-anti-drug-antibody value for the specific patient based on the determined effective drug half-life.

16. The method of claim 1, further comprising setting a new dose interval for each of the plurality of available doses of the drug for the specific patient to the plurality of time-to-target values for the specific patient.

17. The method of claim 16, further comprising providing a recommendation to use Bayesian individualized dosing for the specific patient, in an event the new dose interval is less than a standard-of-care dose interval.

18. The method of claim 16, further comprising treating the specific patient for one of IBD, RA, JIA, AS, PsO, PsA, MS, atopic dermatitis, eczema, and asthma, with an intravenous or subcutaneous administration of the new dosage of the monoclonal antibody or the monoclonal antibody construct at the new dose interval.

19-43. (canceled)
